SEQUENCE LISTING (27 sequences)

SEQ ID NO: 1 - DNA, 363 bp, synthetic construct
  source 1..363: mol_type = other DNA
caggtgcagctggtggagtctgggggaggcgtgatccagcctgggaggtccctgagactctcctgtgcagcgtctggattcaccttcagtgcgtatcttatgcactgggtccgccaggctccaggcaaggggctggagtgggtggcagctataggttatgatggaatgaataaatactatgcagactccgtgaagggccgattcaccatctccagagacaattccaagaacacgctgtatctgcaaatgaacagcctgagagccgaggacacggcggtgtactactgcgccagagctggaatagaatacagctacgcctatactgattactggggacagggtacattggtcaccgtctcctca

SEQ ID NO: 2 - DNA, 327 bp, synthetic construct
  source 1..327: mol_type = other DNA
gaaattgtgttgacgcagtctccaggcaccctgtctttgtctccaggggaaagagccaccctctcctgcagggccagtcagttcgtttacagcagcgacttagcctggtaccagcagaaacctggccaggctcccaggctcctcatctatggtgcatccgccagggccgccggcatcccagacaggttcagtggcagtgggtctgggacagacttcactctcaccatcagcagactggagcctgaagattttgcagtgtattactgtatgcagttcggatggtggcctcctaggactttcggcggagggaccaaggttgagatcaaa

SEQ ID NO: 3 - protein, 121 aa, synthetic construct
QVQLVESGGGVIQPGRSLRLSCAASGFTFSAYLMHWVRQAPGKGLEWVAAIGYDGMNKYYADSVKGRFTISRDNSKNTLYLQMNSLRAEDTAVYYCARAGIEYSYAYTDYWGQGTLVTVSS

SEQ ID NO: 4 - protein, 109 aa, synthetic construct
EIVLTQSPGTLSLSPGERATLSCRASQFVYSSDLAWYQQKPGQAPRLLIYGASARAAGIPDRFSGSGSGTDFTLTISRLEPEDFAVYYCMQFGWWPPRTFGGGTKVEIK

SEQ ID NO: 5 - protein, 9 aa, synthetic construct
FTFSAYLMH

SEQ ID NO: 6 - protein, 17 aa, synthetic construct
AIGYDGMNKYYADSVKG

SEQ ID NO: 7 - protein, 14 aa, synthetic construct
ARAGIEYSYAYTDY

SEQ ID NO: 8 - protein, 12 aa, synthetic construct
RASQFVYSSDLA

SEQ ID NO: 9 - protein, 7 aa, synthetic construct
GASARAA

SEQ ID NO: 10 - protein, 10 aa, synthetic construct
MQFGWWPPRT

SEQ ID NO: 11 - protein, 9 aa, synthetic construct
GSLSGYPWS

SEQ ID NO: 12 - protein, 16 aa, synthetic construct
ETDVSGWTDYNPSLKS

SEQ ID NO: 13 - protein, 14 aa, synthetic construct
ARDGRRMGTPSFDI

SEQ ID NO: 14 - protein, 11 aa, synthetic construct
RASQDINSFLA

SEQ ID NO: 15 - protein, 7 aa, synthetic construct
AASSLNS

SEQ ID NO: 16 - protein, 9 aa, synthetic construct
QKSVYFPPT

SEQ ID NO: 17 - protein, 11 aa, synthetic construct
RASQGINSFLA

SEQ ID NO: 18 - protein, 7 aa, synthetic construct
AADSIQS

SEQ ID NO: 19 - protein, 9 aa, synthetic construct
QKAVYFPPT

SEQ ID NO: 20 - protein, 11 aa, synthetic construct
RASKGISSFLA

SEQ ID NO: 21 - protein, 11 aa, synthetic construct
RASQGISSFLA

SEQ ID NO: 22 - protein, 7 aa, synthetic construct
AASSLQS

SEQ ID NO: 23 - protein, 9 aa, synthetic construct
QSAVYFPPT

SEQ ID NO: 24 - DNA, 1353 bp, synthetic construct
  source 1..1353: mol_type = other DNA
caagttcaactccaacaatggggtgccgggctgctgaagccgtccgaaacgttgagtctcacctgcgccgtgtacggcggcagtctttcaggctatccatggtcctggataaggcagccccccggcaagggtctcgaatggataggtgagacggacgtgtctggctggacagattacaacccttctctgaagtctcgcgttaccatttccgtagatacaagtaagaatcagttctcactgaagctcagttctgtaaccgctgccgacaccgcagtgtactattgtgcaagagatggacgcaggatgggaactccaagctttgacatttggggccagggtacgatggtgactgtttcctccgcctcaacgaaagggccatctgtattcccattggctccgagtagtaagagtacaagtggtgggaccgcagcgctgggatgcctcgtcaaggattattttcccgaaccagtaactgtaagttggaatagcggggcattgacaagcggcgttcacacctttccagcggtgcttcaatcttctggactctactcattgagctcagttgtcaccgttccttcaagttccttgggcacgcagacatatatctgcaatgtgaatcataaaccttctaatacgaaggtcgataagaaagtggagccaaaatcatgtgataagacgcatacttgccccccctgcccagcccctgagctgttggcaggtcccgatgtctttctctttccgcccaagccgaaggatacgcttatgatctccaggactcctgaggtgacttgcgtcgttgtcgacgtatcccacgaagatcctgaggtgaagtttaattggtatgtcgacggagtagaggtgcataatgctaaaacgaaacctcgggaagagcaatataattccacttacagagtagtgagcgttctcacagttttgcatcaggactggcttaacggaaaggagtataaatgtaaggtcagtaacaaagctcttccagctcccgaggagaaaacaatttcaaaggcaaagggccaacctagggaaccgcaggtgtatactctccccccaagccgcgaagaaatgacgaaaaaccaggtgtccctcacttgcctggtcaaaggtttctatccttctgacatagcggtggagtgggagagtaacggccaaccagagaacaattataagaccacgcctccagttctggattccgatggatcattctttctgtactctaaattgacggttgacaaatcccgctggcagcagggaaacgtattttcatgttccgtgatgcatgaagctttgcacaatcactacactcaaaagagtttgagcttgtcaccgggaaagtaa

SEQ ID NO: 25 - DNA, 645 bp, synthetic construct
  source 1..645: mol_type = other DNA
gacattcagatgacgcaaagccccagctctgtctcagcttccgtgggtgatagggtgactattacatgtcgagcctcacaagatattaatagctttcttgcatggtatcagcaaaagccagggaaggcacctaagctcctgatttatgctgcctcttctttgaatagcggggtcccctcccgcttctcaggatctgggtcagggactgacttcacgctgactatatcctcactccaaccagaagatttcgccacttattactgccaaaaatccgtatatttcccgcccacattcggtggtgggacaaaagtggaaatcaagagaactgtcgctgccccatctgttttcatctttccaccgagcgacgaacagctcaaaagcggcactgcgagtgttgtttgtctgctgaataacttctatcccagggaagcaaaggtgcagtggaaggtagacaatgctctgcaatccgggaatagtcaggaatccgtcacggagcaagacagtaaggactccacgtattccttgagtagtacattgaccctcagtaaagcggattacgagaaacacaaagtttacgcttgtgaagtaacgcatcaggggttgtccagtccggttaccaaatccttcaatcggggagagtgttga

SEQ ID NO: 26 - protein, 450 aa, synthetic construct
QVQLQQWGAGLLKPSETLSLTCAVYGGSLSGYPWSWIRQPPGKGLEWIGETDVSGWTDYNPSLKSRVTISVDTSKNQFSLKLSSVTAADTAVYYCARDGRRMGTPSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, synthetic construct
DIQMTQSPSSVSASVGDRVTITCRASQDINSFLAWYQQKPGKAPKLLIYAASSLNSGVPSRFSGSGSGTDFTLTISSLQPEDFATYYCQKSVYFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0170159 A1
(43) Pub. Date: May 29, 2025

(54) COMPOSITIONS COMPRISING STING AGONISTS AND METHODS OF USE FOR TREATING GLIOMA

(71) Applicant: ImmunoGenesis, Inc., Houston, TX (US)

(72) Inventors: Federica PERICLE, Houston, TX (US); Michael A. CURRAN, Houston, TX (US)

(21) Appl. No.: 18/760,608

(22) Filed: Jul. 1, 2024

Related U.S. Application Data
(60) Provisional application No. 63/524,349, filed on Jun. 30, 2023.

(51) Int. Cl. A61K 31/7084; A61K 45/06; A61P 35/00

(52) U.S. Cl. CPC A61K 31/7084; A61K 45/06; A61P 35/00

(57) ABSTRACT

The present disclosure relates generally to the field of medicine, oncology, and immunology. More particularly, it comprises combination therapies including a STING agonist and dual specific antibodies with high affinity for PD-L1 and PD-L2 and further encompasses antibody drug conjugates including STING agonists linked to human dual specific antibodies with high affinity for PD-L1 and PD-L2 and their use in cancer therapies.

### CROSS-REFERENCE TO RELATED APPLICATIONS

This application claims the benefit of U.S. provisional patent application No. 63/524,349, which was filed on Jun. 30, 2023 and is incorporated herein by reference in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is IMGE_025_01US_ST26.xml. The XML file is 27.3 kilobytes, was created on Jul. 1, 2024, and is being submitted electronically via USPTO Patent Center.

### FIELD

The present disclosure relates generally to the field of medicine, oncology, and immunology. More particularly, the invention provides highly active cyclic-di-nucleotide (CDN) immune stimulators that are highly effective in treating gliomas that occur in the brain and in various locations in the nervous system, including the brain stem and spinal column.

### BACKGROUND

Blockade of T cell co-inhibitory receptor PD-1 interaction with its ligand PD-L1 has become a pillar of modern oncology, which is now available even in the first line setting for subsets of melanoma and lung cancer patients (Boussiotis, 2016). Numerous antibodies targeting PD-1 or PD-L1 are currently FDA approved or in clinical trials; however, no agents targeting the second PD-1 ligand, PD-L2, are under development. PD-L2 binds PD-1 with an approximately 5-fold higher affinity than does PD-L1, and, like PD-L1, sends an inhibitory signal which attenuates T cell function (Cheng et al., 2013; Latchman et al., 2001; Lee et al., 2016; Li et al., 2017; Youngnak et al., 2003). If both ligands are present at similar expression levels, PD-L2 would be predicted to outcompete PD-L1 binding to PD-1 (Miao Y R et al., BioRxiv-2020). Historically, PD-L2 was considered to be largely an inducible co-inhibitory molecule with expression limited to the tumor stroma; however, improved detection reagents for PD-L2 have revealed widespread PD-L2 expression both in the tumor microenvironment and on tumor cells themselves (Baptista et al., 2016; Danilova et al., 2016; Derks et al., 2015; Dong et al., 2016; Howitt et al., 2016; Kim et al., 2015; Kim et al., 2015; Nomi et al., 2007; Obeid et al., 2016; Ohigashi et al., 2005; Roemer et al., 2016; Shi et al., 2014; Shin et al., 2015; Xu et al., 2016). Recently, PD-L2 was shown to be an independent predictor of response to the PD-1 antibody pembrolizumab across multiple cancers (Yearley et al., 2017). The inventors have previously identified a dual specific antibody, which is disclosed in PCT International Application Publication No. WO 2019/182,867, which is incorporated herein by reference in its entirety.

The inventors have identified administering a STING agonist of the invention and an antibody of the invention or an ADC comprised of a STING agonist of the invention and an antibody of the invention, wherein the antibody is a highly specific, antibody that selectively binds to PD-L1 and LD-L2 or a dual specific antibody that selectively binds to both PD-L1 and PD-L2 while also exhibiting little to no off-target binding is effective is reducing or ameliorating a tumor cell or treating various types of cancer.

### SUMMARY

The invention generally encompasses methods of treating glioma and related disorders by administering a STING agonist of the invention to a subject in need thereof.

In certain embodiments of the invention, the STING agonist compounds of the invention have a structure of Formula Ia:

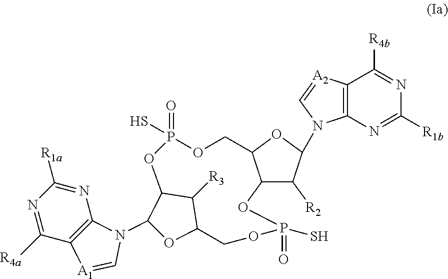

or a salt, ester, hydrate, or solvate thereof, wherein:
A₁ and A₂ are independently selected from CH and N;
R₁ₐ and R_{1b} are independently selected from H and NH₂;
R₂ is selected from OH, F, Cl, N₃, and NH₂;
R₃ is selected from OH, F, Cl, N₃, and NH₂;
R₄ₐ and R_{4b} are independently selected from NH₂, OH, NHR₅, and OR₅; and
R₅ is independently selected from methyl, ethyl, and propyl.

Also included are all stereoisomers, including enantiomers and diastereomers, of compounds of Formula Ia.

In certain embodiments of the present invention, compounds have structural Formula IIa:

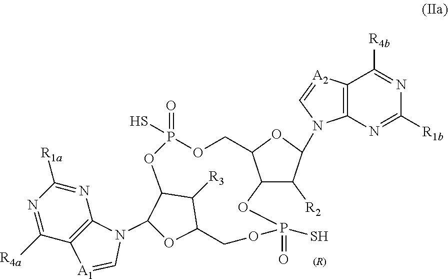

or a salt, ester, tautomer, solvate, hydrate, or prodrug thereof, wherein:
A₁ and A₂ are independently selected from CH and N;
R₁ₐ and R_{1b} are independently selected from H and NH₂;
R₂ is selected from OH, F, and Cl;
R₃ is OH; and
R₄ₐ and R_{4b} are independently selected from NH₂ and OH.

Also provided are stereoisomeric forms of Formula IIa, including enantiomers and diastereomers for which the phosphorus indicated in Formula IIa has R absolute stereochemistry.

In certain embodiments of any of Formulas Ia and IIa, A₁ is CH and A₂ is N.

In certain embodiments of any of Formulas Ia and IIa, A₁ is N and A₂ is CH.

In certain embodiments of any of Formulas Ia and IIa, A₁ and A₂ are both CH.

In certain embodiments of any of Formulas Ia and IIa, A₁ and A₂ are both N.

In certain embodiments of any of Formulas Ia and IIa, A₁ is N and A₂ is CH, and R₂ is selected from F and Cl, and R₃ is-OH.

In certain embodiments of any of Formulas Ia and IIa, A₁ and A₂ are both N, and R₂ is selected from F and Cl, and R₃ is OH.

In certain embodiments of any of Formulas Ia and IIa, R₁ₐ and R_{1b} are both H.

In certain embodiments of any of Formulas Ia and IIa, R₄ₐ and R_{4b} are independently selected from NH₂ and NHR₅.

In certain embodiments of any of Formulas Ia and IIa, R₄ₐ and R_{4b} are independently selected from NH₂ and NHR₅, R₁ₐ and R_{1b} are both H, A₁ and A₂ are both N, R₂ is selected from F and Cl, and R₃ is OH.

In certain embodiments of any of Formulas Ia and IIa, R₄ₐ and R_{4b} are independently selected from NH₂ and NHR₅, R₁ₐ and R_{1b} are both H, A₁ is N and A₂ is CH, R₂ is selected from F and Cl, and R₃ is OH.

In certain embodiments of any of Formulas Ia and IIa, R₄ₐ and R₄ are both NH₂.

In certain embodiments of any of Formulas Ia and IIa, R₄ₐ and R₄ are both NH₂, and R₁ₐ and R_{1b} are both H.

In certain embodiments, the invention encompasses compounds of formula IIIa:

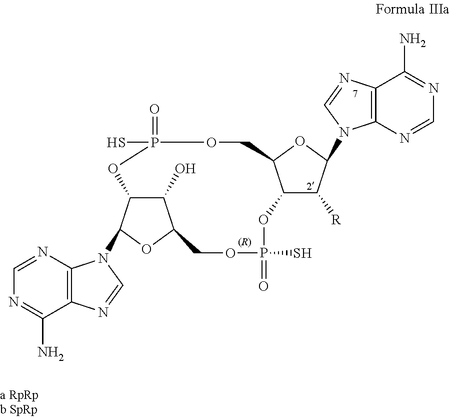

or a salt, ester, hydrate, or solvate thereof, wherein R is F or Cl.

In certain embodiments, the Compound of the Invention is a compound of Formula A with the following structure:

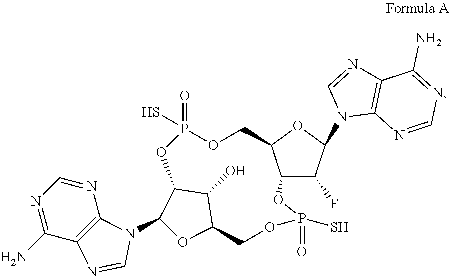 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(N)ncnc21]

or a salt, ester, hydrate, or solvate thereof.

In certain embodiments, the Compound of the Invention is a compound of Formula B with the following structure:

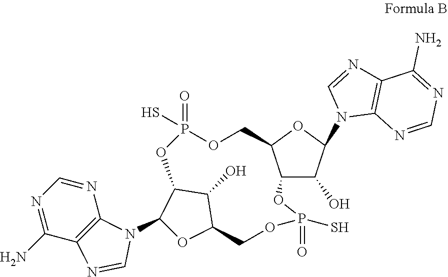 [SMILES: Nc1ncnc2c1ncn2[C@@H]1O[C@@H]2COP(=O)(S)O[C@@H]3[C@H](O)[C@@H](COP(=O)(S)O[C@H]2[C@H]1O)O[C@H]3n1cnc2c(N)ncnc21]

or a salt, ester, hydrate, or solvate thereof.

In certain embodiments, the Compound of the Invention is a compound of Formula C with the following structure:

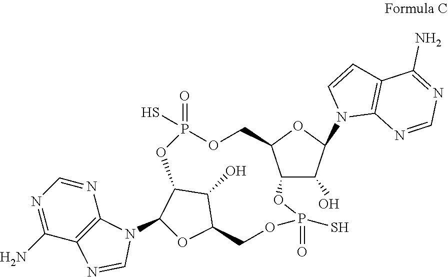 [SMILES: Nc1ncnc2c1ccn2[C@@H]1O[C@@H]2COP(=O)(S)O[C@@H]3[C@H](O)[C@@H](COP(=O)(S)O[C@H]2[C@H]1O)O[C@H]3n1cnc2c(N)ncnc21]

or a salt, ester, hydrate, or solvate thereof.

In certain embodiments, the Compound of the Invention is a compound of Formula D with the following structure:

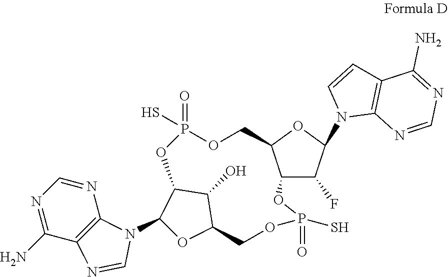 [SMILES: Nc1ncnc2c1ccn2[C@@H]1O[C@@H]2COP(=O)(S)O[C@@H]3[C@H](O)[C@@H](COP(=O)(S)O[C@H]2[C@H]1F)O[C@H]3n1cnc2c(N)ncnc21]

or a salt, ester, hydrate, or solvate thereof.

It will be appreciated by a person in the chemical arts that compounds of Formula (Ia) possess several asymmetric, tetrahedral atoms. Formula (Ia) embraces compounds that possess all possible combinations of absolute stereochemistry at the various asymmetric, tetrahedral atoms. Due to the nonidentical substitution at the two tetrahedral phosphorus atoms, each atom represents a center of chirality. Formula (Ia), (IIa), and (IIIa) include compounds that possess all possible combinations of absolute stereochemistry at the two asymmetric, tetrahedral phosphorus atoms.

In various embodiments, the compounds disclosed herein possess useful STING modulating activity and may be used in the treatment or prophylaxis of glioma or a disorder related to glioma. Thus, in broad aspect certain embodiments also provide pharmaceutical compositions comprising one or more STING agonists disclosed herein together with a pharmaceutically acceptable carrier, as well as methods of making and using the compounds and compositions.

Certain embodiments provide methods for treating glioma by modulating STING comprising administering a STING agonist of the invention to a subject in need thereof. Other embodiments provide methods for treating a glioma or a related disorder in a patient in need of such treatment, comprising administering to said patient a therapeutically effective amount of a STING agonist of the invention.

There is also provided a method of treating glioma with a STING agonist of the invention. As used herein, the term “glioma” refers to tumors that form when glial cells grow out of control. A glioma is a tumor that forms in the brain or spinal cord. There are several types, including astrocytomas, ependymomas and oligodendrogliomas. Some gliomas contain multiple types of cells called mixed gliomas. They categorize each type of glioma as low-, mid- or high-grade based on how fast they grow and other features. Gliomas include: Astrocytomas, including glioblastomas and diffuse intrinsic pontine gliomas (DIPGs): These tumors start in cells called astrocytes. Glioblastomas are astrocytomas that are very aggressive or grow fast. They are the most common malignant brain tumor in adults. Astrocytomas are common gliomas in children. A rare but very aggressive form of brain cancer in children is DIPG. It forms in the brain stem and mostly affects children. Ependymomas: These tumors start in ependymocytes, a type of glial cell. Ependymomas usually form in the ventricles of the brain or the spinal cord. They may spread through cerebrospinal fluid (the fluid that surrounds and protects the brain and spinal cord), but don't spread outside the brain or spine. Ependymomas make up about 2% of all brain tumors. They're more common in children than adults. Oligodendrogliomas: These tumors start in glial cells called oligodendrocytes. Oligodendrogliomas tend to grow more slowly but can become more aggressive over time. Like ependymomas, they rarely spread outside the brain or spine. They're more common in adults than children. Oligodendrogliomas account for about 1% to 2% of all brain tumors.

The methods may further comprise treating glioma in a subject in need thereof with a STING agonist of the invention in combination with another anti-cancer agent or treatment, such as chemotherapy, radiotherapy, immunotherapy, hormonal therapy, or toxin therapy. The second anticancer agent or treatment may be given at the same time as the first agent or given before and/or after the agent.

The invention further encompasses a method of treating glioma wherein myeloid cells within the tumor microenvironment (TME) have prevented effective tumor antigen presentation, suppress both innate and adaptive immune effector responses, and provide ongoing support for glioma stem cell survival and proliferation.

The invention further encompasses a method of treating glioma using a STING agonist of the invention by sensing DNA in the cytoplasm and activating a potent suite of innate immune molecular pathways including NFKB to drive cell-intrinsic inflammation and IRF3 to promote high level secretion of Type 1 Interferons and associated chemokines.

The invention further encompasses a method of treating glioma using a STING agonist of the invention by driving activation of antigen-presenting cells allowing them to provide Signals 1, 2 and 3 for optimal T cell activation. In certain embodiments, STING activation in GBM can repair the broken antigen presentation circuit by mobilizing activated antigen presenting cells to traffic to the cervical lymph node and activate GBM-specific T cells that are then drawn to the tumor in the brain by chemokines produced following STING activation of tumor myeloid cells in situ.

The invention further encompasses a method of treating glioma using a STING agonist of the invention by wiping away immuno-suppressive programming of myeloid stroma (e.g., MDSC, TAM) revealing their underlying pro-inflammatory activity.

The invention further encompasses a method of treating glioma using a STING agonist of the invention. In certain embodiments, multiple checkpoint refractory models of intra-cranial GBM, both focal (QPP8, CT-2A) and diffuse (QPP4) experience curative responses to only 2-3 injections of a Compound of Formula A. In certain embodiments, the activity of the STING agonist sensitizes the GBM to PD-1 blockade.

In various embodiments, the STING agonists of the invention significantly extended survival of U87 tumor bearing animals, wherein no neurological symptoms were noted, and there was no evidence of demyelination. Perivascular lymphocytic infiltrates were not seen which would implicate the induction of autoimmunity. Neuropathologic studies failed to demonstrate either perivascular fibrosis or persistent demyelination

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

The invention also encompasses combination therapies of activation of stimulator of interferon genes (STING) to enhance antitumor immunity. The STING agonists of the invention (e.g., Compounds of the Invention) have been tested for anti-cancer activity in mouse tumor models and in canine companion animals with spontaneous glioblastoma. The STING agonist molecule of the invention increase the efficacy of both PD-L1 and PD-L2 with enhanced ADCC and ADCP effector diminishing the immune suppression in immune excluded tumors.

The invention encompasses administering the STING agonists disclosed herein that used in combination with a anti-PD-L1/PD-L2 monoclonal antibody induces curative responses in checkpoint-refractory tumor models. The improved survival is associated with an increase of T cells and potential decrease of M2 macrophages at the tumor site suggesting that delivery of STING intratumorally can potentiate the systemic activity of a novel checkpoint inhibitor.

In multiple murine glioma models, therapeutic activity was observed with STING agonist 8803 (i.e., Compound A), providing a strong rationale for translational implementation. These models included a humanized mouse model in which the STING pathway has been epigenetically silenced in the glioblastoma cells, recapitulating the biology of human patients. This immunological elimination of glioblastoma is mediated by the orchestrated and cooperative interaction between both the innate and adaptive immune components. The observations that STING agonists upregulate co-stimulation and downmodulate M2 polarization while enhancing T and NK cell proliferation, effector responses, and reducing exhaustion from the glioblastoma TME are consistent with the findings of STING agonists described for other malignancies. Our study reports several observations, including that the expression of STING at the tumor-endothelial junction under baseline untreated conditions is inadequate for facilitating immune cell infiltration and propagation throughout the human glioblastoma TME. The spatial confinement of the T cells in the perivascular space is possibly through the regulation of CXCL12. Our data implicate STING pathway expression within endothelial cells as the instigator of T cell inflammatory responses in the glioblastoma TME and is likely one of the reasons behind the limited effects of T cell-targeted therapies.

In the current study, Compound A was directly administered into the glioblastoma, overcoming the limitations of off-target effects, pharmacokinetic clearance, and the blood-brain barrier. Formulation in a hydrogel for sustained delivery or targeting to myeloid cells using nanotherapeutics is currently under development. Because the mechanism of action for 8803 is through the immune system and is not a direct cytotoxic agent, the entire TME does not need to be exposed to this agent for it to mediate an effect. Despite 8803 being directly injected into the glioblastomas within the brain, this was well tolerated, and no neurological toxicities were observed. When the CNS was evaluated with Luxol Fast Blue, there was no evidence of induced autoimmunity. This may be because the STING pathway is not activated/present within the normal brain vasculature.

The therapeutic effect of 8803 was also found in the QPP8 murine model. This glioblastoma cell line does not have epigenetic silencing of the STING pathway, which would suggest that strategies that demethylate STING in the glioblastoma cell population may have synergy with STING agonists. As there are differences in the methylation of the STING promoter between humans and mice, the preclinical murine studies may be an overestimate of the therapeutic impact of STING agonists. In the case of the humanized murine model with U87, the noted increase in survival could be mediated through tissue rejection through MHC incompatibility, reflecting a tumor rejection paradigm. However, we have previously shown that a closely related 8803 analog, 8779, induces radiographic regression in spontaneously arising glioblastomas in dogs whose glioblastomas are associated with a high abundance of macrophages within the TME (12). The promoter methylation status of STING in canine glioblastoma is unknown. Cumulatively, our study and others indicate that even in malignancies that lack STING expression such as glioblastoma, the myeloid and endothelial stroma may mediate the in vivo responses.

Other strategies for activating the STING pathway include the conjugation of a CpG oligodeoxynucleotide to a targeting moiety. One example is a STING agonist-targeting CD47 antibody that could induce M1 polarization in TAMs, reduce immunosuppression, and inhibit orthotopic glioblastoma by phagocytosis of macrophages and microglia (31). Because there is expression of CD47 on the tumor cells in the CD45-compartment, a substantial fraction of this drug would be shuttled to the tumor cell in which the STING pathway is shut off secondary to STING promoter methylation. A STING agonist strategy affecting a broad range of tumor, immune, and non-immune cells may have a greater therapeutic impact. The high expression of the STING pathway within the myeloid compartment throughout the glioblastoma TME indicates that whether 8803 is injected into the myeloid-enriched tumor itself or the surgical bed infiltrated with microglia, this pathway can be therapeutically modulated. Therapeutic activity can be further enhanced when used in combination with anti-PD-1 in some scenarios in which there is a predisposition to respond. As anti-PD-1 has already received FDA approval and has been used more extensively in glioblastoma (49-51), a clinical trial of this combination may be warranted in a subset of glioblastoma patients. Since 8803 would be a first-in-human study, a phase I clinical trial with intratumoral administration of 8803 into recurrent WT glioblastoma patients would first need to be conducted to define an MTD/MED. We also intend to conduct a window-of-opportunity analysis to enable immune profiling of posttreatment glioblastoma tissue and to ascertain whether STING promoter methylation status correlates with radiographic responses to 8803.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A, 1B, 1C, and 1D collectively illustrate STING agonist (i.e., a Compound of Formula A), dual functioning PD-L1/PD-L2 antibody (27907) and their mechanisms of action. FIG. 1A) Structure of the 2′,3′-linked phosphorothioate diadenosine monophosphate (2′,3′-S2-CDA)-Formula A. FIG. 1B) Overview of the STING pathway (J Clin Invest DOI: 10.1172/JCI86892). STING activation leads to activation of TBK1 and IRF3, leading to production of type I IFNs. FIG. 1C) Graphical representation of 27907 monoclonal antibody. Each antigen binding site is capable of binding either PD-L1 or PD-L2 and blocking their interactions with PD-1. Point mutations within the Fc region enhance the cell-mediated effector functions of the antibody. FIG. 1D) Representative mechanisms of 27907.

FIGS. 2A, 2B, and 2C collectively illustrate exemplary STING agonists of the invention activates STING to produce Type I IFN response in antigen presenting cells.

FIG. 3 illustrates the neurosurgical administration of exemplary compound, 8779

FIG. 4 illustrates a complete radiographic response of the contrast enhancing mass in dog after 2 injections of an exemplary STING agonist of the invention.

FIG. 5 illustrates the doses, volumetric analyses, and outcomes for dogs enrolled in the clinical trial.

FIG. 6 illustrates inflammatory response in unresected high-grade gliomas at various time intervals.

FIG. 7 illustrates localized inflammatory response in unresected high-grade gliomas.

FIGS. 8A and 8B collectively illustrate the activity of the Compound of Formula A and other synthetic and natural STING agonists against reporter cells expressing human (FIG. 8A) or mouse (FIG. 8B) STING. diABZI is a synthetic non-cyclic dinucleotide. 2′3′-c-di-AM (PS) 2 (Rp, Rp) is structurally identical to ADU-S100. c-di-GMP Fluo is a fluorinated analog of bacterial cyclic-diguanylate-GMP. 3′3′-cGAMP Fluo is fluorinated analog of bacterial 3′3′-cGAMP. Linear 2′3′-cGMP is included as a negative control. The concentration of the STING agonist is displayed on the x-axis and luminescence activity is displayed on the y-axis.

FIGS. 9A, 9B, and 9C collectively illustrate the concentration of Ab-27907, pembrolizumab, or IgG isotype is-displayed on the x-axis and luminescence activity in displayed on the y-axis. Mean±SEM (n=3) is displayed for each concentration of each antibody. A dose range of 50.000-0.015 nM was tested against PD-L1+ cells (FIG. 9A), PD-L2+ cells (FIG. 9B), and a 50:50 mixture of PD-L1+ and PD-L2+ cells (FIG. 9C). Nonlinear regression was used to generate best-fit curves in GraphPad Prism.

FIGS. 10A, 10B, 10C, 10D, 10E, and 10F collectively illustrate the concentration of Ab-27907, rituximab, trastuzumab, or IgG isotype is displayed on the x-axis and luminescence activity in displayed on the y-axis. Mean±SEM (n=2) is displayed for each concentration of each antibody. A dose range was tested in ADCC (FIGS. 10A, 10C, and 10E) and ADCP FIGS. 10B, 10C, and 10F) assays against U2940 human B-cell lymphoma (FIGS. 10A and 10B), 8505C human thyroid carcinoma (FIGS. 10C and 10D), and B16F10-PDL2 murine melanoma (FIGS. 10E, and 10F) tumor cell lines.

FIGS. 11A, 11B, and 11C collectively illustrate the Dose dependent effect of 8803 (i.e., the Compound of Formula A) in co-culture systems. FIG. 11A) Luminescence, reporting IRF3 activation, is displayed on the y-axis and 8803 concentration is displayed on the x-axis. FIG. 11B) Luminescence, reporting total tumor cell numbers, is displayed on the y-axis and 8803 concentration is displayed on the x-axis. Healthy donor PMBCs were incubated with 8505C human thyroid carcinoma tumor cells at a ratio of 2:1 for this experiment. FIG. 11C) Optical density, reporting concentration of CXCL10, is displayed on the y-axis and concentration of 8803 is displayed on the x-axis. Supernatant from the experiment shown in B was tested in an ELISA assay.

FIGS. 12A and 12B collectively illustrate tumor growth inhibition (FIG. 12A) and improved survival (FIG. 12B) of B16F10-PDL2 tumor-bearing mice treated with the combination of Compound 8803 and Ab-27907. Left: Mean±SEM tumor volume (mm³) is plotted on the y-axis against days post tumor inoculation on the x-axis. Right: Kaplan Meier plots of survival.

FIGS. 13A, 13B, and 13C collectively illustrate tumor growth inhibition and improved survival of TS/A tumor-bearing mice treated with the combination of Compound 8803 (i.e., Compound of Formula A) and Ab-27907. FIG. 13A: Flow cytometry histograms showing expression of PD-L1 and PD-L2 on TS/A cells. FIG. 13B: Expression of PD-L1 or PD-L2 on mMDSCs, macrophages, and gMDSCs found in TS/A tumors. FIG. 13C: Mean═SEM tumor diameter (mm²) is plotted on the y-axis against days post tumor inoculation on the x-axis. D) Kaplan Meier plots of survival.

FIGS. 14A, 14B, 14C, and 14D collectively illustrate ex vivo analysis of the tumors from TS/A bearing mice treated with Compound 8803 and/or Ab-27907. FIG. 14A) Quantification of necrotic area in excised TS/A tumors untreated, treated with 8803, treated with 27907, or treated with the combination therapy. FIG. 14B) Representative H&E images of excised TS/A tumors.

FIG. 14C) Quantification of CD3+ cells within the TS/A tumors that were untreated, treated with Compound 8803 (i.e., Compound of Formula A), treated with Ab-27907, or treated with the combination. FIG. 14D) Quantification of Foxp3+ cells identified within the TS/A tumors that were untreated, treated with 8803, treated with 27907, or treated with the combination.

FIGS. 15A, 15B, 15C, 15D, 15E, 15F, 15G, and 15H collectively illustrate the therapeutic effect of STING agonist 8803 (i.e., Compound A) in humanized glioma mouse model recapitulating human glioblastoma. (FIG. 15A) Multiplexed sequential immunofluorescence images of the STING expression at baseline in humanized mouse brain implanted with U87 glioma cells and collected at endpoint. The right image represents a high magnification of the white box drawn in the left image. The white arrows highlight CD31+STING+ vessels and green arrows highlight the CD163+STING+ macrophages. DAPI (dark blue), GFAP (light blue), CD31 (cyan blue), STING (red), CD163 (green). Scale bars: 500 μm (left panel) and 50 μm (right panel). A higher magnification image of a CD163+STING+ cell is represented at the upper right quadrant of the right image (scale bar: 20 μm). (FIG. 15B) Humanized mice that underwent i.e. implantation of 112.5×103 (survival) or 90× 103 (immune infiltrate analysis) human U87 glioma cells treated with PBS (n=18), the moderately potent STING agonist MLRR-S2-CDA (n=19), or 8803 (n=12) on days 5, 10, and 15. The survival rate of the humanized mice was estimated by the Kaplan-Meier method. Control: median survival (MS): 26.5 days, MLRR-S2-CDA MS: 35 days, 8803 MS: 43.5 days. Statistics: control versus MLRR-S2-CDA log-rank **** P<0.0001; control versus 8803 log-rank **** P<0.0001. (FIG. 15C) Luxol Fast Blue demonstrating uniform staining without evidence of clearance that would be reflective of demyelination in the CNS in either the control or 8803-treated brains (×1.5 magnification). (FIG. 15D) Representative hematoxylin and eosin-stained coronal sections of mice at the survival endpoint demonstrating persistent glioma after treatment with 8803. Original magnification, ×1.25 (left and middle images) and ×10 (bottom right image). (FIG. 15E, 15F) Ex vivo Flow cytometric analysis of U87-infiltrating human immune cells using BD LSRFortessa X-30 prototype flow cytometer. MFI, mean fluorescence intensity; h, human; TAM, tumor-associated macrophage; Mono, monocyte; PMN, peripheral mononuclear cell; MDSC, myeloid-derived suppressor cell. (FIG. 15G, 15H) WT C57BL/6J mouse bone marrow-derived macrophages pretreated with IL-4 for 48 hours followed by STING agonist 8803 for the indicated times (24 hours and 48 hours). The markers were assessed via Cytek Aurora flow cytometer and the CD45+CD11b+ population was analyzed for the indicated markers. *P<0.05; ** P<0.01; *** P<0.001; **** P<0.0001 by 2-tailed Student's t test (FIGS. 15E-H).

FIGS. 16A, 16B, 16C, 16D, 16E, 16F, 16G, and 16H collectively illustrate the therapeutic effect of the STING agonist 8803 (Compound A) in immune checkpoint blockade-resistant preclinical models of glioblastoma. FIG. 16A, 16B: Treatment of immunocompetent C57BL/6J mice with i.e. implantation of either QPP4 (FIG. 16A) or QPP8 (FIG. 16B) glioma cells. The survival rate of C57BL/6J mice with i.e. implanted QPP4 treated with 8803 (5 μg) on days 14 and 28 (n=16) significantly prolonged survival relative to vehicle control mice (n=16; median survival [MS]=72 days; log-rank P=0.0003). Similarly, both 8803 and 8879 (n=17; undefined MS) were curative in the QPP8 model (control group: n=15; MS=103 days) when they were administered on days 14, 21, and 28 after implantation. Agonist 8803 versus vehicle control (log-rank P<0.0001); 8879 versus vehicle control (log-rank P=0.0002). FIG. 16C, 16D: Flow cytometric analysis of tumor-infiltrating immune cells using BD LSRFortessa X-30 prototype flow cytometry. QPP8 cells were orthotopically implanted in C57BL/6J mice and then treated with PBS or 5 μg 8803 on days 60 and 67. Tumors were isolated 48 hours after the final treatment and immune cells were collected using a Percoll gradient. The total amount of immune cells was quantified based on all live CD45+ cells and specifically on CD11b+Ly6C+ expression of mono-MDSCs. (FIG. 16C) Within the myeloid compartment from the tumor and cervical lymph nodes (LNs), immune cell lineages were identified based on standard surface markers (Supplemental Table 1), and then the mean fluorescence intensity (MFI) was quantified based on treatment. Each dot represents an analyzed tumor or LN. FIG. 16E: Expression of immunosuppressive markers CD206 and CD163 spanning myeloid populations and as a function of treatment. FIG. 16F: Myeloid PD-L1 expression on various immune lineages in tumors and LNs. FIG. 16G: Conventional type 1 DCs (cDC1s) were increased in both tumor and LNs in response to 8803. FIG. 16H: Immunosuppressive arginase expression spanning myeloid populations and as a function of treatment. * P<0.05; ** P<0.01; *** P<0.001; **** P<0.0001 by 2-tailed Student's t test (FIG. 16C-16H).

FIGS. 17A, 17B, 17C, 17D, 17E, 17F, and 17G collectively illustrate STING agonist 8803 (Compound A) induces immune effector responses within gliomas. Flow cytometric analysis of QPP8-infiltrating immune cells using BD LSRFortessa X-30 prototype flow cytometer. QPP8 cells were orthotopically implanted into C57BL/6J mice and then treated with PBS or 5 μg 8803 on days 60 and 67. Tumors were isolated 48 hours after the final treatment, and immune cells were collected using a Percoll gradient. FIG. 17A: Within the CD8+ T cell compartment from the tumor and cervical lymph nodes (LNs), 8803 enhanced the number of infiltrating CD8+ T cells. FIG. 17B: CD8+ T cell immune exhaustion markers such as PD-1 and LAG-3 were decreased, but proliferation and granzyme B expression increased. FIG. 17C: NK cell infiltration and frequency and granzyme B expression were increased in 8803-treated gliomas. FIG. 17D: The survival rate estimated by the Kaplan-Meier method of RAG−/−versus WT C57BL/6J mice implanted with QPP8v (subclone) and treated with STING agonist 8803 versus PBS. RAG−/−control (PBS): 5 mice (median survival [MS]: 63 days); WT control: 5 mice (MS: 76 days); RAG−/−8803:5 mice (MS: 55 days); WT 8803:5 mice (MS: undefined; 3 long-term survivors). Statistics (log-rank test): RAG−/−control versus WT control P=0.209; RAG−/−control versus RAG−/−8803 P=0.192; RAG−/−control versus WT 8803 P=0.0018; RAG−/−8803 versus WT 8803 P=0.0018; RAG−/−8803 versus WT control P=0.014; WT control versus WT 8803 P=0.0018. FIG. 17E: The survival estimated by the Kaplan-Meier method of C57BL/6J mice implanted with QPP8v and treated with either 8803 or the combination of 8803+NK1.1 antibody (aNK1.1). Control (PBS): 5 mice (MS: 34 days); 8803:5 mice (MS: 76 days); 8803+aNK1.1:5 mice (MS: 79 days). Statistics (log-rank test): control versus 8803 P=0.0017; control versus 8803+aNK1.1 P=0.0062; 8803 versus 8803+aNK1.1 P=0.92. FIG. 17F, 17G: Expansion of OT-1 CD8+ T cells or WT CD8+ T cells collected from spleen and then treated with PBS versus 8803 at 1, 5, and 10 μM. * P<0.05; ** P<0.01; *** P<0.001; **** P<0.0001 by 2-tailed Student's t test (A-C) or 2-way ANOVA (FIG. 17G).

FIGS. 18A, 18B, 18C, 18D, and 18E collectively illustrate dose and schedule adjustments for combinatorial antibody with 8803: combination with anti-PD-1. FIG. 18A: Schema of the treatment of immunocompetent mice that underwent i.e. implantation of either GL261 or CT-2A glioma cells. FIG. 18B: The survival rate estimated by the Kaplan-Meier method of C57BL/6J mice implanted with GL261 (left panel) and CT-2A (right panel). For the GL261 survival: control: 10 mice (median survival [MS]: 25 days), WP1066: 10 mice (MS: 30 days), 8803:14 mice (MS: 30 days; 5 long-term survivors), WP1066+8803:16 mice (MS: 71 days; 8 long-term survivors). Statistics (log-rank test): control versus WP1066 P=0.04; control versus 8803 P=0.01; control versus WP1066+8803 P<0.0001; WP1066 versus WP1066+8803 P=0.006; 8803 versus WP1066+8803 P=0.32. For the CT-2A survival: control: 10 mice (MS: 28 days), WP1066: 9 mice (MS: 28 days), 8803:14 mice (MS: 50 days; 7 long-term survivors), WP1066+8803:16 mice (MS: 48 days; 8 long-term survivors). Statistics (log-rank test): control versus WP1066 P=0.57; control versus 8803 P<0.0001; control versus WP1066+8803 P<0.0001; WP1066 versus WP1066+8803 P<0.001; 8803 versus WP1066+8803 P=0.95. FIG. 18C: Schema of the treatment of immunocompetent mice that underwent i.e. implantation of either CT-2A or QPP8v (subclone) glioma cells. QPP8v tumors received 8803 (5 μg/mouse i.e. on days 7 and 17), anti-PD-1 (25 μg i.e. on days 7 and 17), and vehicle control (PBS) or anti-PD-1 (250 μg i.p. on days 7, 10, and 13) in single and combination therapies. (FIG. 18D) The survival rate estimated by the Kaplan-Meier method of C57BL/6J mice implanted with CT-2A (left panel) and QPP8 (right panel). For CT-2A: IgG: 10 mice (MS: 30 days); anti-PD-1:10 mice (MS: 35 days), 8803:10 mice (MS: 56 days, 4 long-term survivors); 8803+ anti-PD-1:10 mice (MS: undefined, 8 long-term survivors); 8803+IgG: 10 mice (MS: 65.5 days, 5 long-term survivors). Statistics (log-rank test): IgG versus anti-PD-1 P=0.04; IgG versus 8803 P<0.01; IgG versus 8803+anti-PD-1 P<0.0001; anti-PD-1 versus 8803+anti-PD-1 P<0.001; 8803 versus 8803+anti-PD-1 P=0.04; 8803+IgG versus 8803+anti-PD-1 P=0.12. For QPP8: PBS: 10 mice (MS: 47 days); anti-PD-1:10 mice (MS: 55 days); 8803:10 mice (MS: 76 days, 4 long-term survivors); 8803+anti-PD-1 (i.p.): 10 mice (MS: 95.5 days, 3 long-term survivors); 8803+anti-PD-1 (i.e.): 10 mice (MS: 111 days, 5 long-term survivors). Statistics (log-rank test): PBS versus anti-PD-1 P=0.164; PBS versus 8803 P<0.0001; PBS versus 8803+anti-PD-1 (i.p.) P=0.0006; PBS versus 8803+anti-PD-1 (i.e.) P<0.0001; anti-PD-1 versus 8803 P<0.0001; anti-PD-1 versus 8803+anti-PD-1 (i.p.) P=0.002; anti-PD-1 versus 8803+anti-PD-1 (i.e.) P<0.0001; 8803 versus 8803+anti-PD-1 (i.e.) P=0.57; 8803 versus 8803+anti-PD-1 (i.p.) P=0.85. FIG. 18E: Multiplexed sequential immunofluorescence images of untreated CT-2A gliomas (n=3). Forty-eight hours after either the first (day 9) or second dose (day 16) of 8803, animals were euthanized and the brains were imaged for the following (n=3/group): DAPI (dark blue), GFAP (light blue), CD31 (cyan blue), STING (red), p-IRF3 (pink), CD4+ (yellow), and CD8+ (white) T cells. White boxes outline the portion shown at higher magnification in the right column of images. Scale bars: 500 μm (left 3 columns) and 50 μm (right column).

FIGS. 19A and 19B collectively illustrate STING agonist 8803 (i.e., Compound A) anti-tumor activity enhancement by IMGS-001. Animals bearing bilateral flank B16-F10 tumors were treated with the STING agonist 8803 and/or anti-PD-1 (Ab-38002). The mice were randomized to receive (i) anti-PD-1 (10 mg/kg/mouse are administered i.p. 2 times per week for 3 weeks); (ii) STING agonist 8803 (5 μg/mouse are administered i.p. once per week for 2 weeks; (iii) Ab-38002-10 mg/kg, i.p, on day 13, 2 times/week for 3 weeks+STING agonist 8803, 10 mg, i.t., day 13 (3 times per day for 3-4 days).

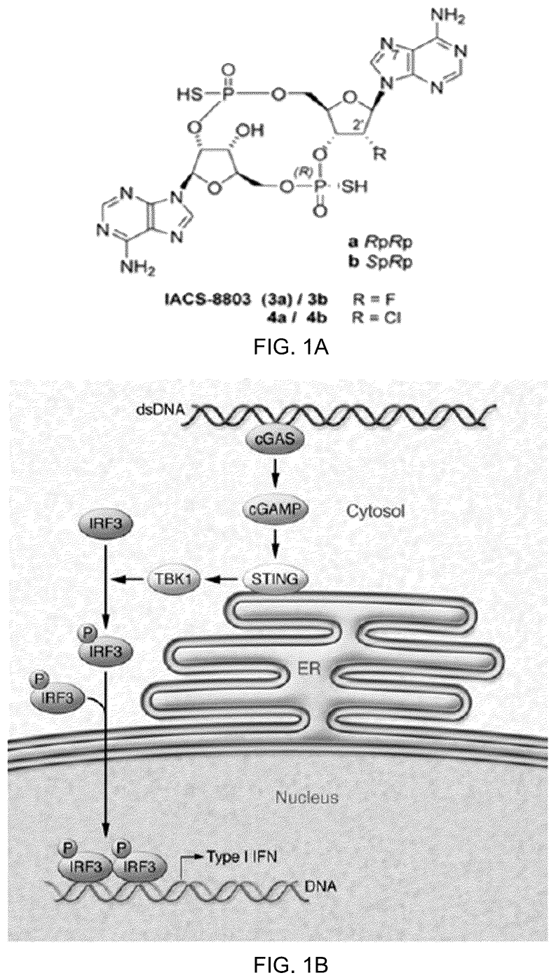

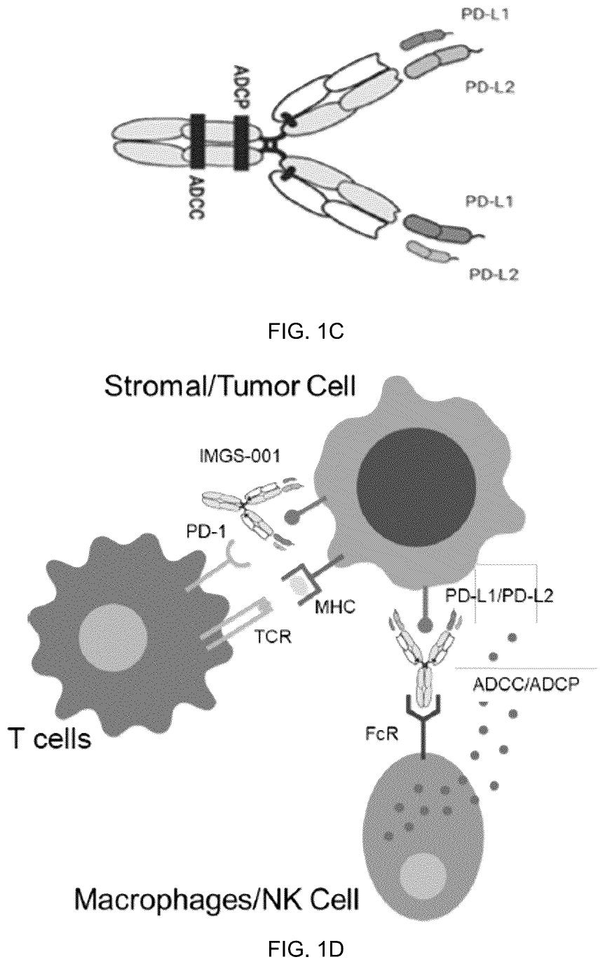

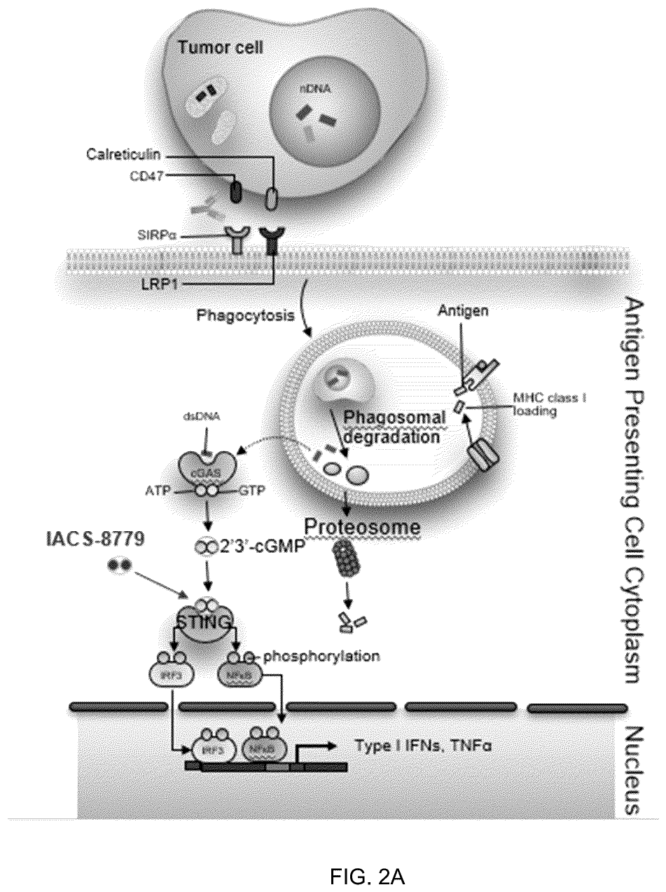

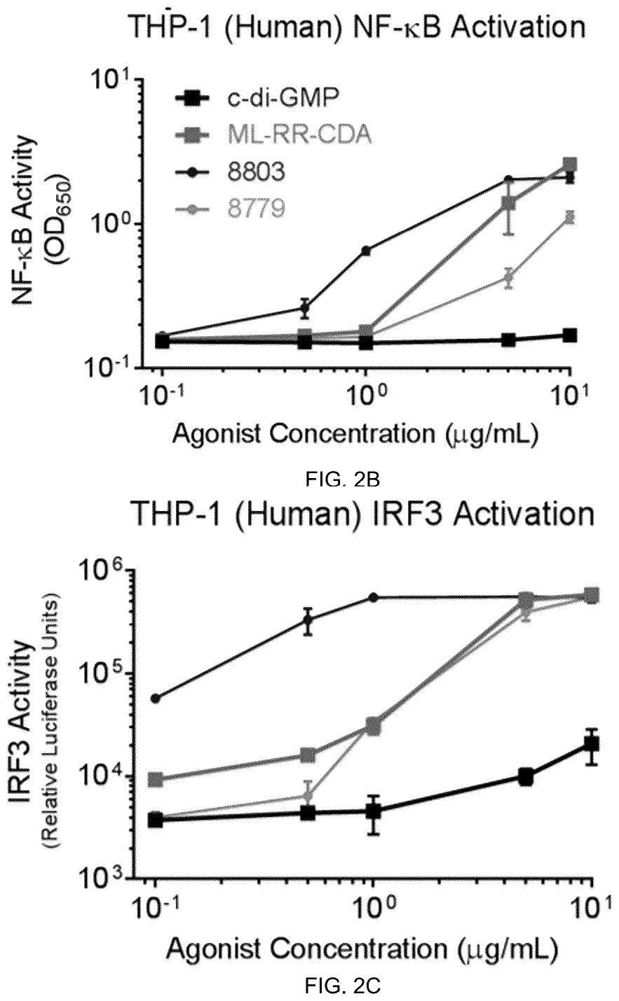

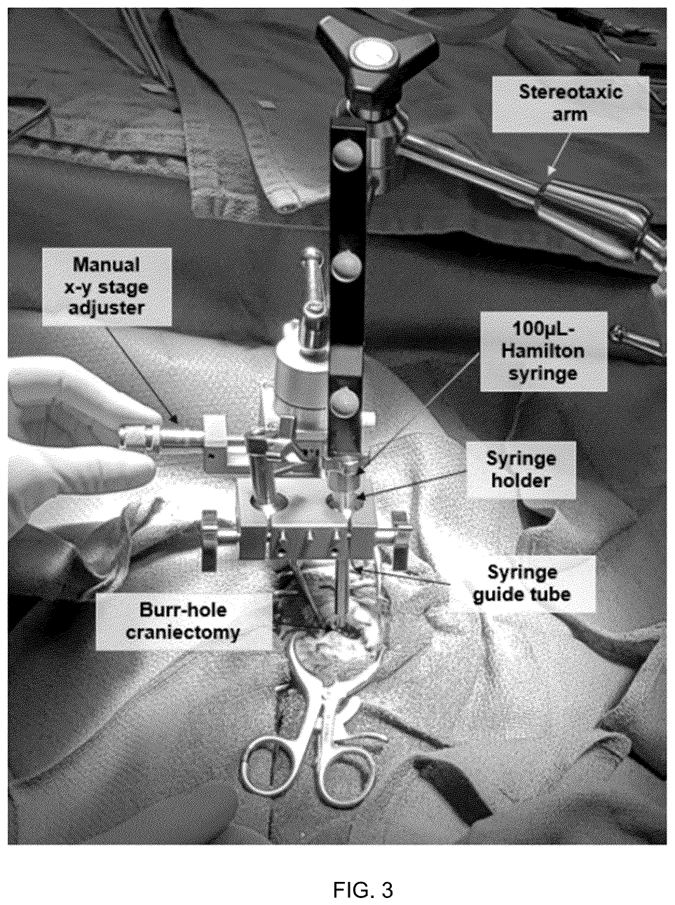

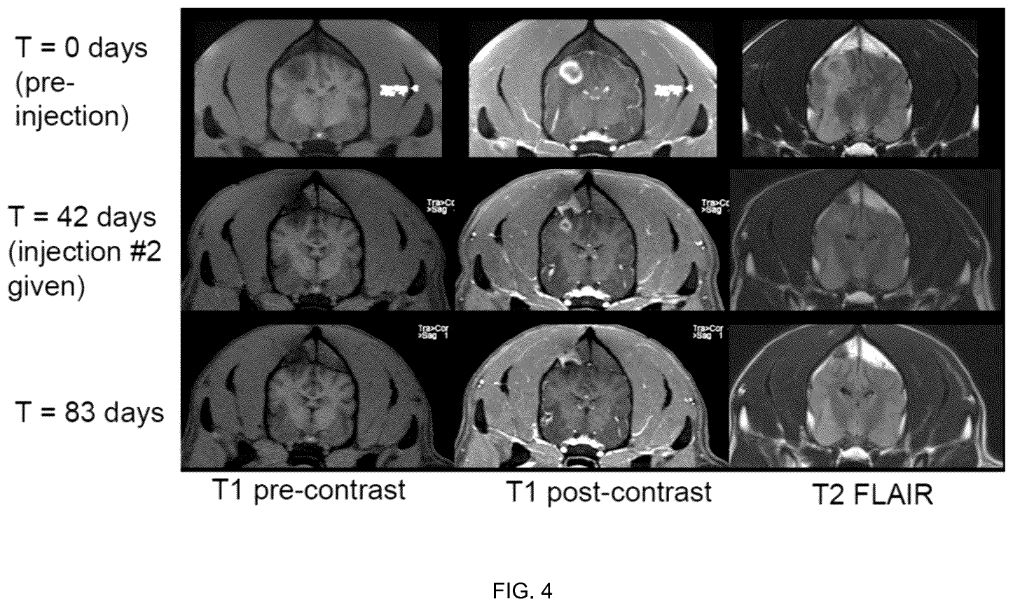

### DETAILED DESCRIPTION OF THE INVENTION

The invention encompasses methods of treating glioma in a subject in need thereof by administering a STING agonist for treating and preventing diseases and disorders including for ameliorating tumors and treating cancer related to glioma.

### I. STING Agonists of the Invention

The invention encompasses cyclic dinucleotides are agonists of the receptor STING for the treatment of gliomas. The term “agonist” refers to any substance that activates a biologic receptor in vitro or in vivo to provoke a physiologic response.

“STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”. STING is a transmembrane receptor protein that in humans is encoded by the gene TMEM173. Activation of STING by cyclic dinucleotides (CDN) leads to activation of the IRF3 and NF-kappa.B pathways and consequently, to induction of Type I interferons and of pro-inflammatory cytokines, respectively. In response to viral infection, STING activates STAT6 (signal transducer and activator of transcription 6) to induce (Th2-type), increase (IL-12) or decrease (IL-10) production of various cytokines, including the chemokines CCL2, CCL20, and CCL26 (Chen et al., 2011).

The term “STING agonist” herein refers to a substance that activates the receptor STING in vitro or in vivo. According to the invention, a compound is deemed to be a STING agonist if: it induces Type I interferons in vitro in human or animal cells that contain active STING; and it does not induce Type I interferons in vitro in human or animal cells that do not contain active STING.

A typical test to ascertain whether a ligand is a STING agonist is to incubate the ligand in a wild-type human or animal cell line and in the corresponding cell line in which the STING coding gene has been genetically inactivated by small or long base deletions (e.g. a homozygous STING knockout cell line). An agonist of STING will induce Type I interferons in the wild-type cells but will not induce Type I interferons in the cells in which the STING coding gene has been inactivated.

The cyclic dinucleotides of the invention induce Type I interferons in vitro in human or animal cells that contain active STING. However, they do not induce Type I interferons in vitro in human or animal cells that do not contain active STING.

The present invention is concerned with fluorinated deoxyribo-cyclic dinucleotides (CDNs). Specifically, it is concerned with (3′,3′)-2′ (mono- or di-fluorinated)-2′-deoxyribo-(CDNs).

The STING agonist compounds of the invention have a structural Formula Ia:

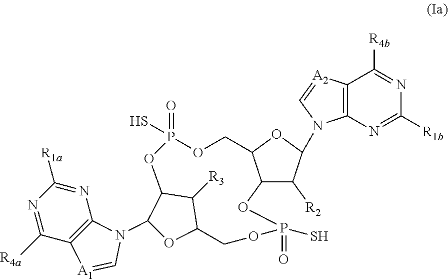

or a salt, ester, hydrate, solvate, tautomer, or prodrug thereof, wherein:
A₁ and A₂ are independently selected from CH and N;
R₁ₐ and R_{1b} are independently selected from H and NH₂;
R₂ is selected from OH, F, CI, N₃, and NH₂;
R₃ is selected from OH, F, CI, N₃, and NH₂;
R₄ₐ and R_{4b} are independently selected from NH₂, OH, NHR₅, and OR₅; and
R₅ is independently selected from methyl, ethyl, and propyl.

Also provided are all stereoisomers, including enantiomers and diastereomers, of compounds of Formula Ia.

In certain embodiments of the present invention, the STING agonist compounds have the structural Formula IIa:

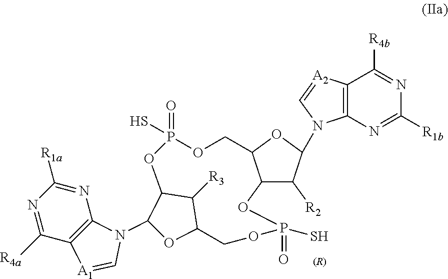

or a salt, ester, hydrate, solvate, tautomer, or prodrug thereof, wherein:
A₁ and A₂ are independently selected from CH and N;
R₁ₐ and R_{1b} are independently selected from H and NH₂;
R₂ is selected from OH, F, and Cl;
R₃ is OH; and
R₄ₐ and R_{4b} are independently selected from NH₂ and OH.

Also provided are stereoisomeric forms of Formula IIa, including enantiomers and diastereomers for which the phosphorus indicated in Formula IIa has R absolute stereochemistry.

In certain embodiments of any of Formulas Ia and IIa, A₁ is CH and A₂ is N.

In certain embodiments of any of Formulas Ia and IIa, A₁ is N and A₂ is CH.

In certain embodiments of any of Formulas Ia and IIa, A₁ and A₂ are both CH.

In certain embodiments of any of Formulas Ia and IIa, A₁ and A₂ are both N.

In certain embodiments of any of Formulas Ia and IIa, A₁ is N and A₂ is CH, and R₂ is selected from F and Cl, and R₃ is OH.

In certain embodiments of any of Formulas Ia and IIa, A₁ and A₂ are both N, and R₂ is selected from F and Cl, and R₃ is OH.

In certain embodiments of any of Formulas Ia and IIa, R₁ₐ and R_{1b} are both H.

In certain embodiments of any of Formulas Ia and IIa, R₄ₐ and R_{4b} are independently selected from NH₂ and NHR₅.

In certain embodiments of any of Formulas Ia and IIa, R₄ₐ and R_{4b} are independently selected from NH₂ and NHR₅, R₁ₐ and R_{1b} are both H, Aj and A₂ are both N, R₂ is selected from F and Cl, and R₃ is OH.

In certain embodiments of any of Formulas Ia and IIa, R₄ₐ and R_{4b} are independently selected from NH₂ and NHR₅, R₁ₐ and R_{1b} are both H, A₁ is N and A₂ is CH, R₂ is selected from F and Cl, and R₃ is OH.

In certain embodiments of any of Formulas Ia and IIa, R₄ₐ and R_{4b} are both NH₂.

In certain embodiments of any of Formulas Ia and IIa, R₄ₐ and R₄ are both NH₂, and R₁ₐ and R_{1b} are both H.

Illustrative embodiments of STING agonists of the invention include the following structures illustrated in Table 2.

TABLE 2

| No. | Structure |
|---|---|
| 101 | 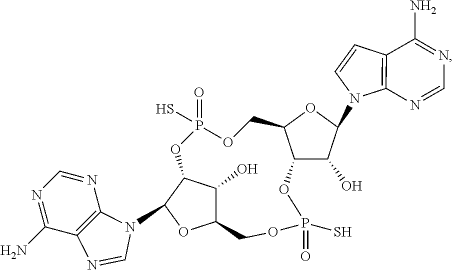 [SMILES: Nc1ncnc2c1ccn2[C@@H]1O[C@@H]2COP(=O)(S)O[C@@H]3[C@H](O)[C@@H](COP(=O)(S)O[C@H]2[C@H]1O)O[C@H]3n1cnc2c(N)ncnc21] |
| 102 | 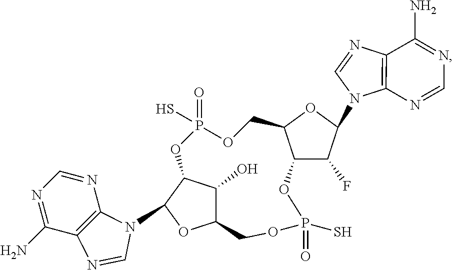 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(N)ncnc21] |
| 103 | 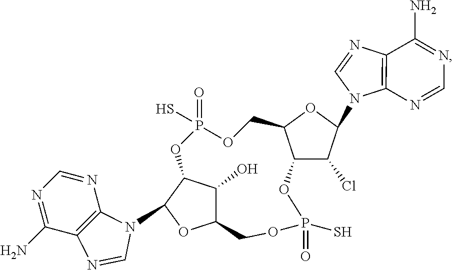 [SMILES: Nc1ncnc2c1ncn2[C@@H]1O[C@@H]2COP(=O)(S)O[C@@H]3[C@H](O)[C@@H](COP(=O)(S)O[C@H]2[C@H]1Cl)O[C@H]3n1cnc2c(N)ncnc21] |
| 104 | 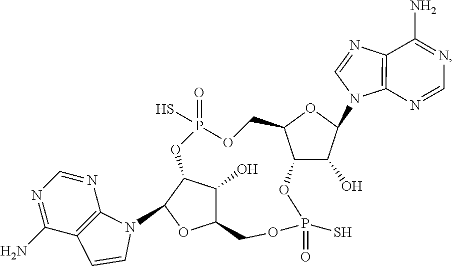 [SMILES: Nc1ncnc2c1ccn2[C@@H]1O[C@@H]2COP(=O)(S)O[C@H]3[C@@H](O)[C@H](n4cnc5c(N)ncnc54)O[C@@H]3COP(=O)(S)O[C@@H]1[C@@H]2O] |
| 105 | 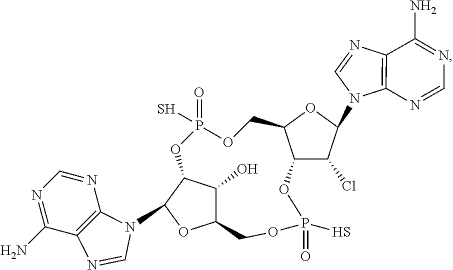 [SMILES: Nc1ncnc2c1ncn2[C@@H]1O[C@@H]2COP(=O)(S)O[C@@H]3[C@H](O)[C@@H](COP(=O)(S)O[C@H]2[C@H]1Cl)O[C@H]3n1cnc2c(N)ncnc21] |
| 106 | 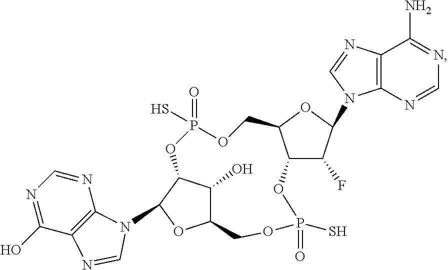 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(O)ncnc21] |
| 107 | 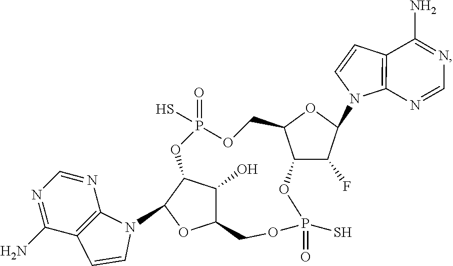 [SMILES: Nc1ncnc2c1ccn2[C@@H]1O[C@@H]2COP(=O)(S)O[C@@H]3[C@H](O)[C@@H](COP(=O)(S)O[C@H]2[C@H]1F)O[C@H]3n1ccc2c(N)ncnc21] |
| 108 | 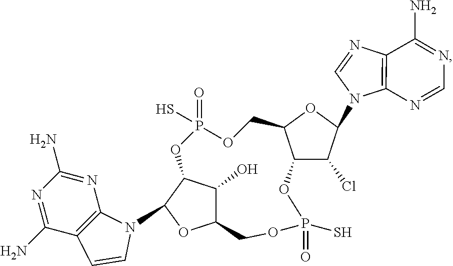 [SMILES: Nc1nc(N)c2ccn([C@@H]3O[C@@H]4COP(=O)(S)O[C@H]5[C@@H](Cl)[C@H](n6cnc7c(N)ncnc76)O[C@@H]5COP(=O)(S)O[C@@H]3[C@@H]4O)c2n1] |
| 109 | 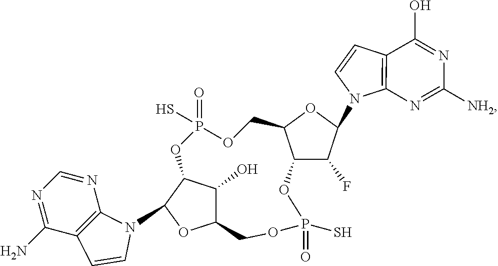 [SMILES: Nc1nc(O)c2ccn([C@@H]3O[C@@H]4COP(=O)(S)O[C@@H]5[C@H](O)[C@@H](COP(=O)(S)O[C@H]4[C@H]3F)O[C@H]5n3ccc4c(N)ncnc43)c2n1] |
| 110 | 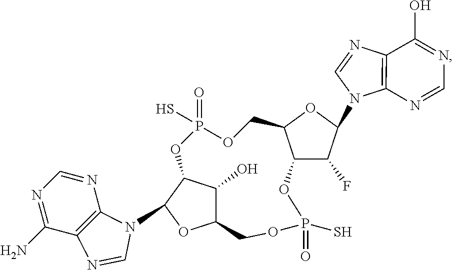 [SMILES: Nc1ncnc2c1ncn2[C@@H]1O[C@@H]2COP(=O)(S)O[C@H]3[C@@H](F)[C@H](n4cnc5c(O)ncnc54)O[C@@H]3COP(=O)(S)O[C@@H]1[C@@H]2O] |
| 111 | 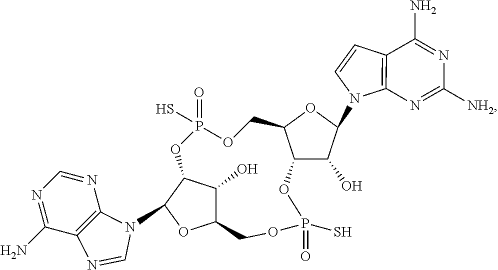 [SMILES: Nc1nc(N)c2ccn([C@@H]3O[C@@H]4COP(=O)(S)O[C@@H]5[C@H](O)[C@@H](COP(=O)(S)O[C@H]4[C@H]3O)O[C@H]5n3cnc4c(N)ncnc43)c2n1] |
| 112 | 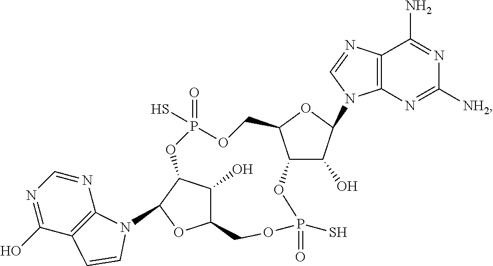 [SMILES: Nc1nc(N)c2ncn([C@@H]3O[C@@H]4COP(=O)(S)O[C@@H]5[C@H](O)[C@@H](COP(=O)(S)O[C@H]4[C@H]3O)O[C@H]5n3ccc4c(O)ncnc43)c2n1] |
| 113 | 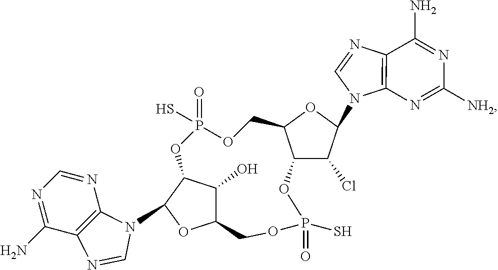 [SMILES: Nc1nc(N)c2ncn([C@@H]3O[C@@H]4COP(=O)(S)O[C@@H]5[C@H](O)[C@@H](COP(=O)(S)O[C@H]4[C@H]3Cl)O[C@H]5n3cnc4c(N)ncnc43)c2n1] |
| 114 | 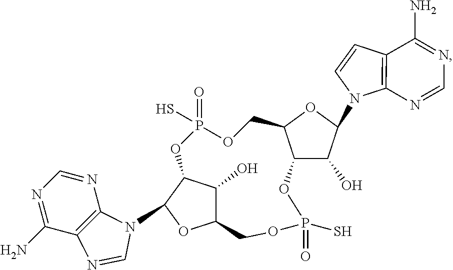 [SMILES: Nc1ncnc2c1ccn2[C@@H]1O[C@@H]2COP(=O)(S)O[C@@H]3[C@H](O)[C@@H](COP(=O)(S)O[C@H]2[C@H]1O)O[C@H]3n1cnc2c(N)ncnc21] |
| 115 | 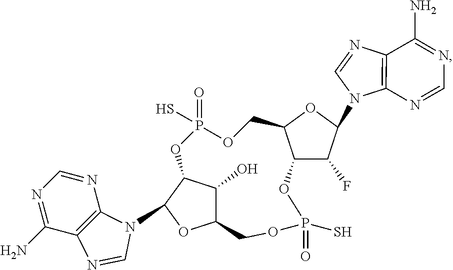 [SMILES: Nc1ncnc2c1ncn2[C@@H]1O[C@@H]2COP(=O)(S)O[C@@H]3[C@H](O)[C@@H](COP(=O)(S)O[C@H]2[C@H]1F)O[C@H]3n1cnc2c(N)ncnc21] |
| 116 | 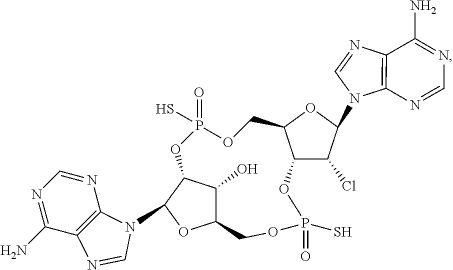 [SMILES: Nc1ncnc2c1ncn2[C@@H]1O[C@@H]2COP(=O)(S)O[C@@H]3[C@H](O)[C@@H](COP(=O)(S)O[C@H]2[C@H]1Cl)O[C@H]3n1cnc2c(N)ncnc21] |
| 117 | 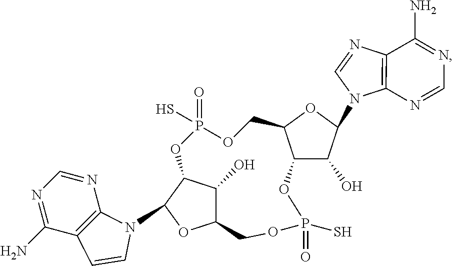 [SMILES: Nc1ncnc2c1ccn2[C@@H]1O[C@@H]2COP(=O)(S)O[C@H]3[C@@H](O)[C@H](n4cnc5c(N)ncnc54)O[C@@H]3COP(=O)(S)O[C@@H]1[C@@H]2O] |
| 118 | 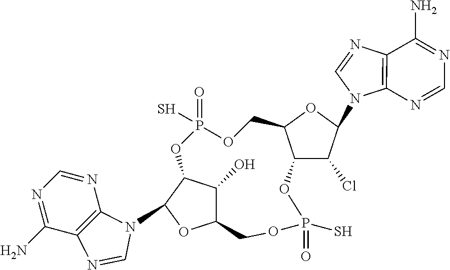 [SMILES: Nc1ncnc2c1ncn2[C@@H]1O[C@@H]2COP(=O)(S)O[C@@H]3[C@H](O)[C@@H](COP(=O)(S)O[C@H]2[C@H]1Cl)O[C@H]3n1cnc2c(N)ncnc21] |
| 119 | 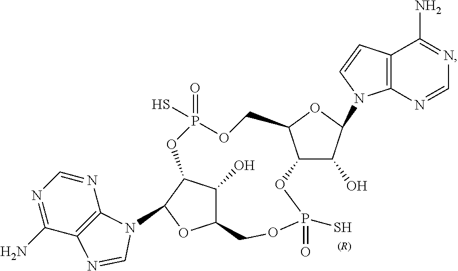 |
| 120 | 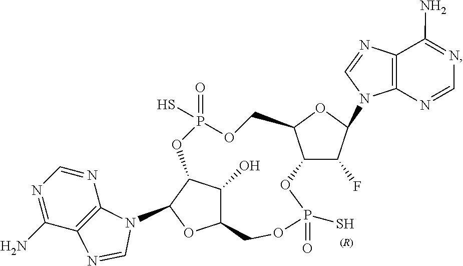 |
| 121 | 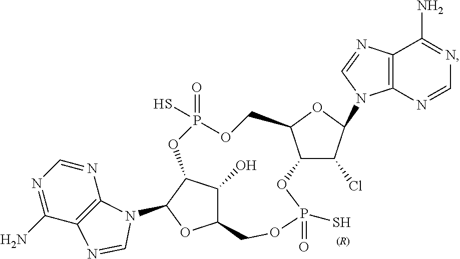 |
| 122 | 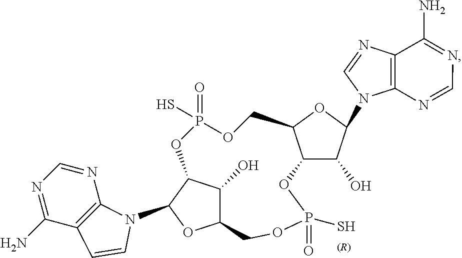 |
| 123 | 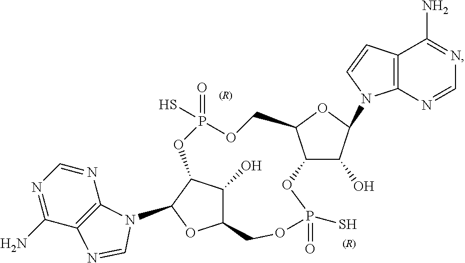 |
| 124 | 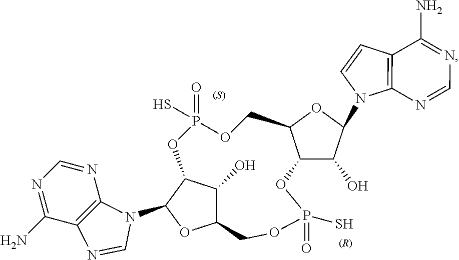 |
| 125 | 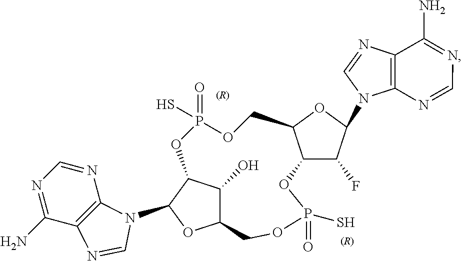 |
| 126 | 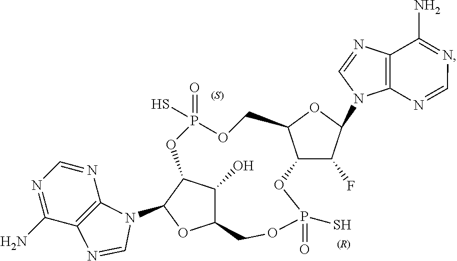 |
| 127 | 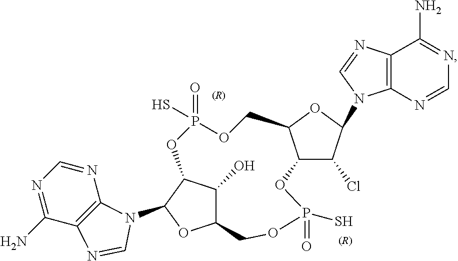 |
| 128 | 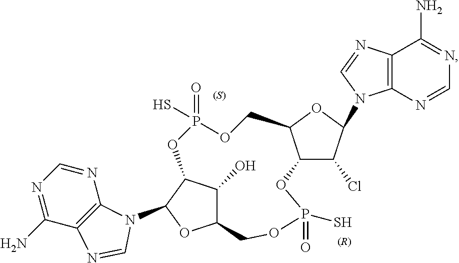 |
| 129 | 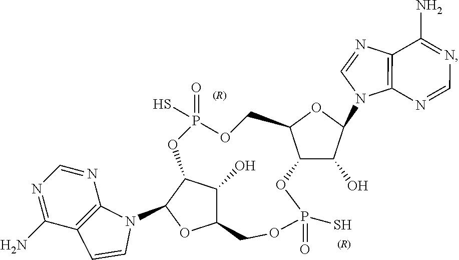 |
| 130 | 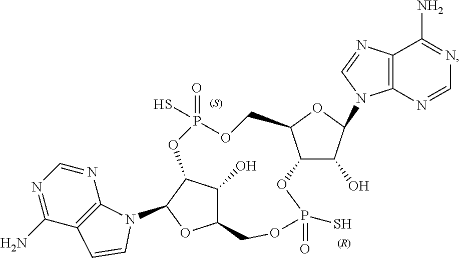 |
| 131 | 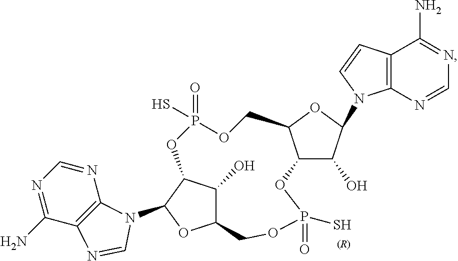 |
| 132 | 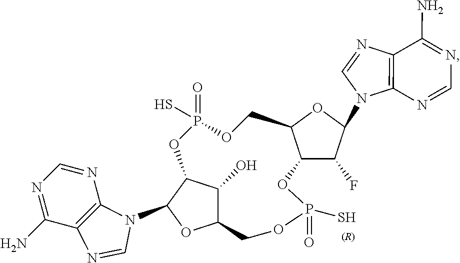 |
| 133 | 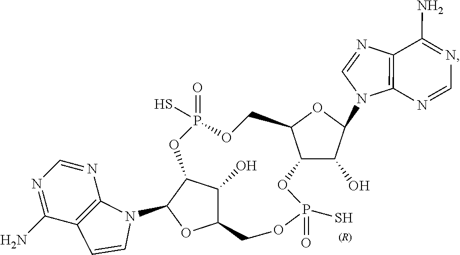 |
| 134 | 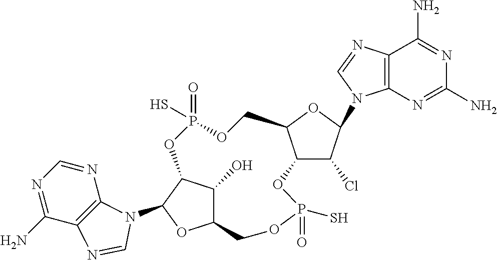 [SMILES: Nc1nc(N)c2ncn([C@@H]3O[C@@H]4CO[P@@](=O)(S)O[C@@H]5[C@H](O)[C@@H](COP(=O)(S)O[C@H]4[C@H]3Cl)O[C@H]5n3cnc4c(N)ncnc43)c2n1] |
| 135 | 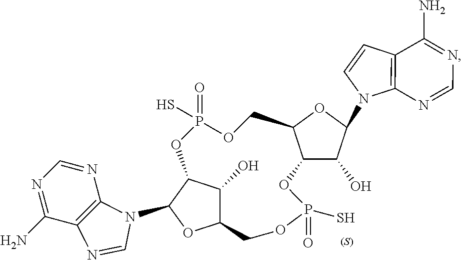 |
| 136 | 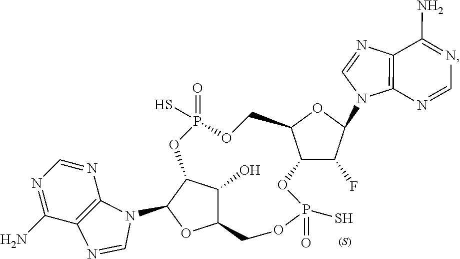 |
| 137 | 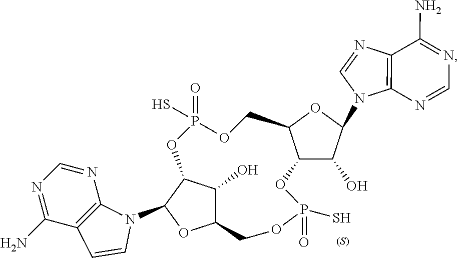 |
| 138 | 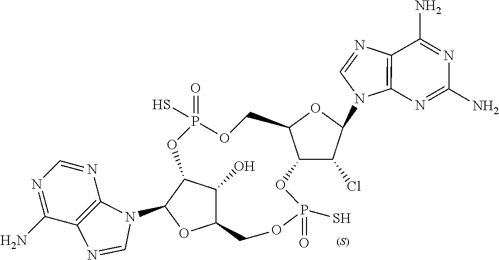 |
| 139 | 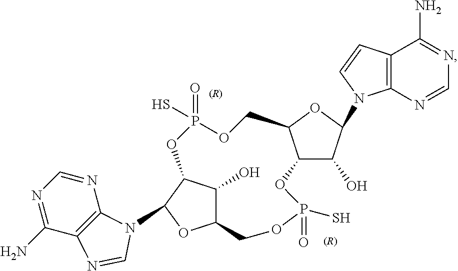 |
| 140 | 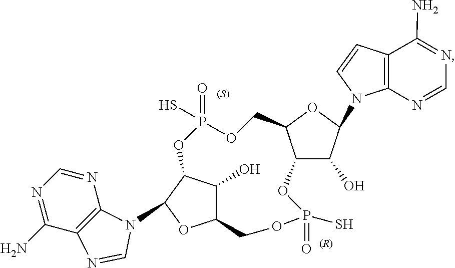 |
| 141 | 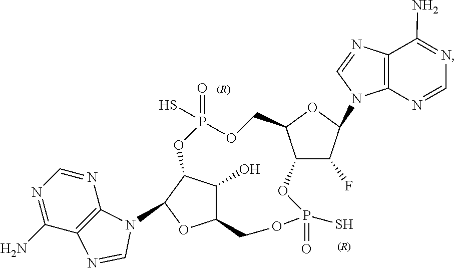 |
| 142 | 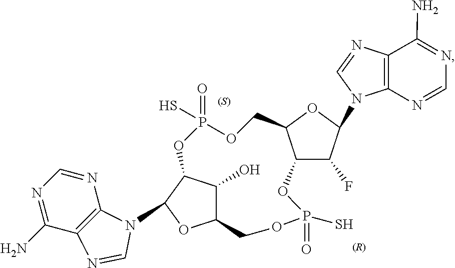 |
| 143 | 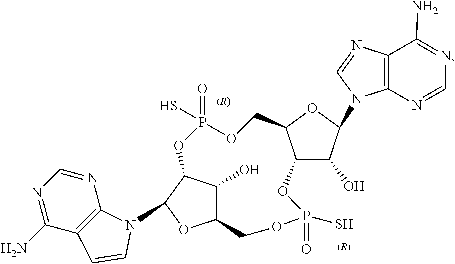 |
| 144 | 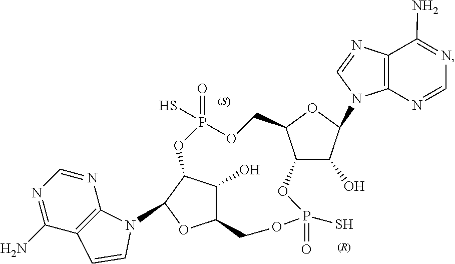 |
| 145 | 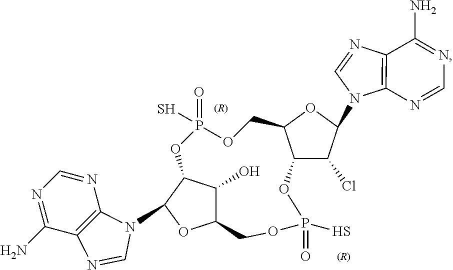 |
| 146 | 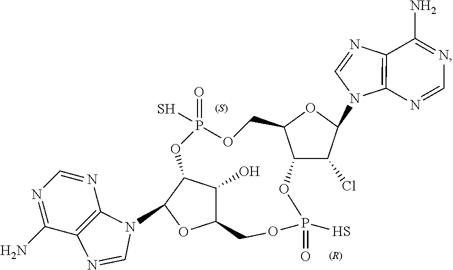 |
| 147 | 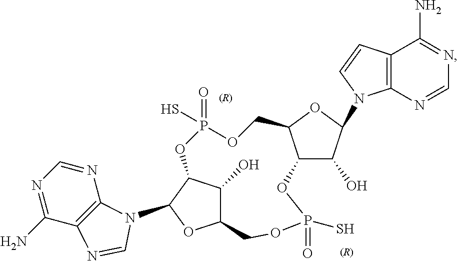 |
| 148 | 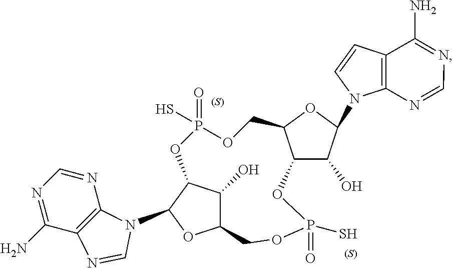 |
| 149 | 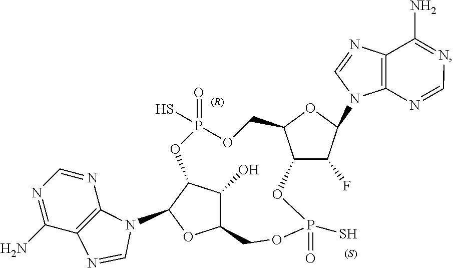 |
| 150 | 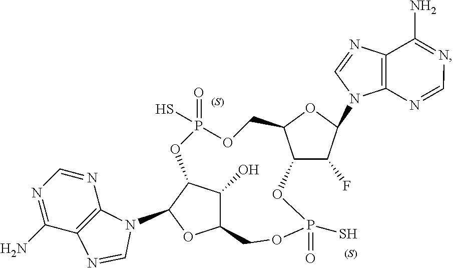 |
| 151 | 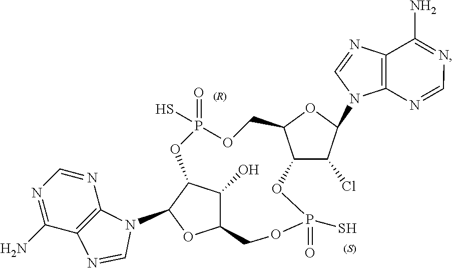 |
| 152 | 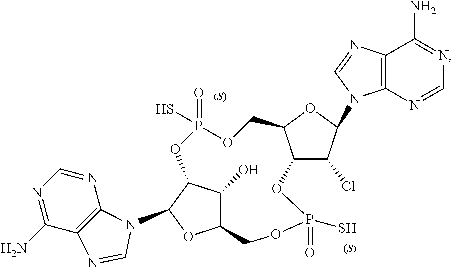 |
| 153 | 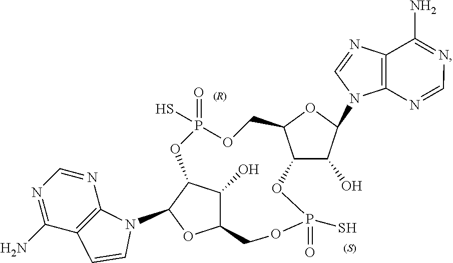 |
| 154 | 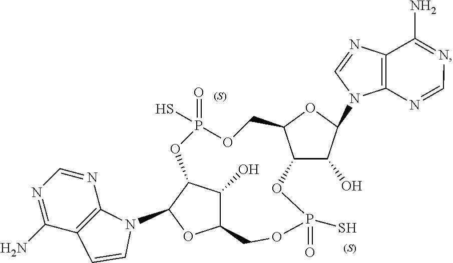 |
| 155 | 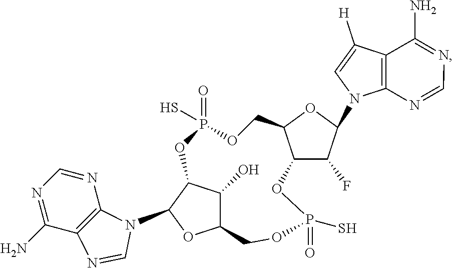 [SMILES: Nc1ncnc2c1ccn2[C@@H]1O[C@@H]2CO[P@@](=O)(S)O[C@@H]3[C@H](O)[C@@H](CO[P@@](=O)(S)O[C@H]2[C@H]1F)O[C@H]3n1cnc2c(N)ncnc21] |

Also encompassed by the invention is a compound chosen from the Examples, or a salt, ester, tautomer, or prodrug thereof.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH2 is mutually exclusive with an embodiment wherein the same group is NH.

### II. Pharmaceutical Formulations and Treatment of Cancer

### A. Cancers

In various embodiments, the invention includes compositions comprising one or more STING agonists of the invention. In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition includes a therapeutically effective amount of a STING agonist of the invention and is non-toxic, does not cause side effects, or both.

“Therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of tumor size.

The STING agonists of the invention can be administered to animals by any number of suitable administration routes or formulations. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of the STING agonists of the invention can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., cancer), and the severity of the disease (e.g., stage or severity of cancer). Of course, combinations of administration routes can be administered, as desired.

Some embodiments of the invention include a method for providing a subject with a composition comprising one or more STING agonists of the invention described herein (e.g., a pharmaceutical composition) which comprises one or more administrations of one or more such compositions; the compositions may be the same or different if there is more than one administration.

In some embodiments, cancers that can be treated in an animal (e.g., mammals, porcine, canine, avian (e.g., chicken), bovine, feline, primates, rodents, monkeys, rabbits, mice, rats, and humans) using a STING agonists of the include, but are not limited to, CNS cancer (e.g., glioblastoma, glioblastoma multiforme, gliosarcoma, or astrocytoma), glioblastoma, glioblastoma multiforme, glioma, gliosarcoma, malignant nerve sheath tumors, medulloblastoma, meningioma, multiple myeloma, neuroblastoma, cancers that can result in metastasis, cancers resulting from metastasis, or cancerous tumors thereof.

As used herein, the term “subject” refers to both human and animal subjects. In some instances, the animal is in need of the treatment (e.g., by showing signs of disease or cancer, or by having a cancerous tumor).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating CNS cancer (e.g., glioblastoma, glioblastoma multiforme, gliosarcoma, or astrocytoma), treating can include but is not limited to prophylactic treatment and therapeutic treatment. As such, treatment can include, but is not limited to: preventing CNS cancer (e.g., glioblastoma, glioblastoma multiforme, gliosarcoma, or astrocytoma).

Treatment of an human can occur using any suitable administration method (such as those disclosed herein) and using any suitable amount of a STING agonists of the invention. In some embodiments, methods of treatment comprise treating an animal for cancer (e.g., CNS cancer (e.g., glioblastoma, glioblastoma multiforme, gliosarcoma, or astrocytoma). Some embodiments of the invention include a method for treating a subject (e.g., an animal such as a human or primate) with a composition comprising a STING agonists of the invention (e.g., a pharmaceutical composition) which comprises one or more administrations of one or more such compositions; the compositions may be the same or different if there is more than one administration.

In some embodiments, the method of treatment includes administering an effective amount of a composition comprising a STING agonists of the invention. As used herein, the term “effective amount” refers to a dosage or a series of dosages sufficient to affect treatment (e.g., to treat cancer, such as but not limited to CNS cancer (e.g., glioblastoma, glioblastoma multiforme, gliosarcoma, or astrocytoma). In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of at least one STING agonists of the invention (which can be administered to an animal such as mammals, primates, monkeys or humans) can be an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight or about 6.5 mg/kg human body weight.

In some instances, an effective amount of at least one STING agonist of the invention (which can be administered to an animal such as mammals, rodents, mice, rabbits, feline, porcine, or canine) can be an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of at least one compound of the invention (which can be administered to an animal such as mammals, primates, monkeys or humans) can be an amount of about 1 μg/kg to about 1000 mg/kg body weight, about 5 μg/kg to about 500 mg/kg body weight, about 10 μg/kg to about 200 mg/kg body weight, about 25 μg/kg to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 20 mg/kg human body weight or about 100 mg/kg human body weight.

In some embodiments, the treatments can also include one or more of surgical intervention, chemotherapy, radiation therapy, hormone therapies, immunotherapy, and adjuvant systematic therapies. Adjuvants may include but are not limited to chemotherapy (e.g., temozolomide), radiation therapy, antiangiogenic therapy (e.g., bevacizumab), and hormone therapies, such as administration of LHRH agonists; antiestrogens, such as tamoxifen; high-dose progestogens; aromatase inhibitors; and/or adrenalectomy. Chemotherapy can be used as a single-agent or as a combination with known or new therapies.

In some embodiments, the administration of at least one STING agonist of the invention is an adjuvant cancer therapy or part of an adjuvant cancer therapy. Adjuvant treatments include treatments by the mechanisms disclosed herein and of cancers as disclosed herein, including, but not limited to tumors. Corresponding primary therapies can include, but are not limited to, surgery, chemotherapy, or radiation therapy. In some instances, the adjuvant treatment can be a combination of chemokine receptor antagonists with traditional chemotoxic agents or with immunotherapy that increases the specificity of treatment to the cancer and potentially limits additional systemic side effects. In still other embodiments, a STING agonist of the invention can be used as adjuvant with other chemotherapeutic agents. The use of a STING agonist of the invention can, in some instances, reduce the duration of the dose of both drugs and drug combinations reducing the side effects.

In some embodiments, the treatments disclosed herein can include use of other drugs (e.g., antibiotics) or therapies for treating disease. For example, antibiotics can be used to treat infections and can be combined with a compound of the invention to treat disease (e.g., infections associated with cancer). In other embodiments, intravenous immunoglobulin (IVIG) therapy can be used as part of the treatment regime (i.e., in addition to administration of the compound(s) of the invention).

In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

### B. Formulation and Administration

The present disclosure provides pharmaceutical compositions comprising a STING agonist of the invention. In addition, the administration of the STING agonist may be administered by different routes of administration. For example, the STING agonist can be administered intratumorally.

The invention also includes pharmaceutical compositions comprising a STING agonist. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

### C. Combination Therapies

In the context of the present disclosure, it also is contemplated that the STING agonist described herein could be used similarly in further combination with chemo- or radiotherapeutic intervention, or other treatments.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with a STING agonist according to the disclosure and at least one other agent. This may be achieved by contacting the cell with a single composition or pharmacological formulation that includes both agents, or by contacting the cell with two distinct compositions or formulations, at the same time.

Administration of the therapeutic agents of the invention to a patient will follow general protocols for the administration of that particular secondary therapy, taking into account the toxicity, if any, of the antibody treatment. It is expected that the treatment cycles would be repeated as necessary. It also is contemplated that various standard therapies, as well as surgical intervention, may be applied in combination with the described cancer therapies.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### 1. Chemotherapy

Cancer therapies also include a variety of combination therapies with both chemical and radiation-based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

### 2. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### 3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

An alternative aspect of immunotherapy is to anticancer effects with immune stimulatory effects. Immune stimulating molecules also exist including cytokines such as IL-2, IL-4, IL-12, GM-CSP, gamma-IFN, chemokines such as MIP-1, MCP-1, IL-8 and growth factors such as FL T3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons, and; IL-1, GM-CSP and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses antitumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders was few compared to those who did not respond.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989). Possible therapeutic antibodies include anti-TNF, anti-CD25, anti-CD3, anti-CD20, CTLA-4-IG, and anti-CD28.

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

### 4. Antibodies of the Invention

The invention also an antibody or antibody fragment that binds selectively to both PD-L1 and PD-L2 and having clone-paired heavy and light chain CDR sequences from Table 3. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% or greater identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% or greater identity to clone-paired sequences from Table 2.

TABLE 1
Nucleic Acid Sequences For Antibody
Variable Regions

| Antibody | Sequence |
|---|---|
| 27907 | CAGGTGCAGCTGGTGGAGTCTGGGGGAGGCGTGATCCAGC |
| Heavy | CTGGGAGGTCCCTGAGACTCTCCTGTGCAGCGTCTGGATT |
| BiPDL4-3₄ | CACCTTCAGTGCGTATCTTATGCACTGGGTCCGCCAGGCT |
| SEQ ID | CCAGGCAAGGGGCTGGAGTGGGTGGCAGCTATAGGTTATG |
| NO: 1 | ATGGAATGAATAAATACTATGCAGACTCCGTGAAGGGCCG |
| | ATTCACCATCTCCAGAGACAATTCCAAGAACACGCTGTAT |
| | CTGCAAATGAACAGCCTGAGAGCCGAGGACACGGCGGTGT |
| | ACTACTGCGCCAGAGCTGGAATAGAATACAGCTACGCCTA |
| | TACTGATTACTGGGGACAGGGTACATTGGTCACCGTCTCC |
| | TCA |
| 27907 | GAAATTGTGTTGACGCAGTCTCCAGGCACCCTGTCTTTGT |
| Light | CTCCAGGGGAAAGAGCCACCCTCTCCTGCAGGGCCAGTCA |
| BiPDL4-3₄ | GTTCGTTTACAGCAGCGACTTAGCCTGGTACCAGCAGAAA |
| SEQ ID | CCTGGCCAGGCTCCCAGGCTCCTCATCTATGGTGCATCCG |
| NO: 2 | CCAGGGCCGCCGGCATCCCAGACAGGTTCAGTGGCAGTGG |
| | GTCTGGGACAGACTTCACTCTCACCATCAGCAGACTGGAG |
| | CCTGAAGATTTTGCAGTGTATTACTGTATGCAGTTCGGAT |
| | GGTGGCCTCCTAGGACTTTCGGCGGAGGGACCAAGGTTGA |
| | GATCAAA |

TABLE 2
Protein Sequences for Variable Regions

| Antibody | Sequence |
|---|---|
| 27907 | QVQLVESGGGVIQPGRSLRLSCAASGFTFSAYLMHWV |
| Heavy | RQAPGKGLEWVAAIGYDGMNKYYADSVKGRFTISRDN |
| BiPDL4-3₄ | SKNTLYLQMNSLRAEDTAVYYCARAGIEYSYAYTDYW |
| SEQ ID NO: 3 | GQGTLVTVSS |
| 27907 | EIVLTQSPGTLSLSPGERATLSCRASQFVYSSDLAWY |
| Light | QQKPGQAPRLLIYGASARAAGIPDRFSGSGSGTDFTL |
| BiPDL4-3₄ | TISRLEPEDFAVYYCMQFGWWPPRTFGGGTKVEIK |
| SEQ ID NO: 4 | |

TABLE 3
CDR Heavy and Light Chain Sequences for Ab-27907

| | CDRH1 | CDRH2 | CDRH3 |
|---|---|---|---|
| Antibody | (SEQ ID NO:) | (SEQ ID NO:) | (SEQ ID NO:) |
| 27907 | FTFSAYLMH | AIGYDGMNKYYA | ARAGIEYSYAYT |
| Heavy | (SEQ ID | DSVKG | DY |
| BIPDL4-3₄ | NO: 5) | (SEQ ID | (SEQ ID |
| | | NO: 6) | NO: 7) |
| 27907 | RASQFVYSSDLA | GASARAA | MQFGWWPPRT |
| Light | (SEQ ID | (SEQ ID | (SEQ ID |
| BIPDL4-3₄ | NO: 8) | NO: 9) | NO: 10) |

In accordance with the present disclosure, there is also provided an antibody or antibody fragment that binds selectively to both PD-L1 and PD-L2 (dual specific antibody to PD-L1 and PD-L2 (DSPDL)) and (i) having heavy chain CDR sequences of CDR1 GSLSGYPWS (SEQ ID NO: 11), CDR2 ETDVSGWTDYNPSLKS (SEQ ID NO: 12), and CDR3 ARDGRRMGTPSFDI (SEQ ID NO: 13) and light chain CDR sequences of CDR1 RASQDINSFLA (SEQ ID NO: 14), CDR2 AASSLNS (SEQ ID NO: 15), and CDR3 QKSVYFPPT (SEQ ID NO: 16); (ii) having heavy chain CDR sequences of CDR1 GSLSGYPWS (SEQ ID NO: 11), CDR2 ETDVSGWTDYNPSLKS (SEQ ID NO: 12), and CDR3 ARDGRRMGTPSFDI (SEQ ID NO: 13) and light chain CDR sequences of CDR1 RASQGINSFLA (SEQ ID NO: 17), CDR2 AADSIQS (SEQ ID NO: 18), and CDR3 QKAVYFPPT (SEQ ID NO: 19); or (iii) having heavy chain CDR sequences of CDR1 GSLSGYPWS (SEQ ID NO: 11), CDR2 ETDVSGWTDYNPSLKS (SEQ ID NO: 12), and CDR3 ARDGRRMGTPSFDI (SEQ ID NO: 13) and light chain CDR sequences of CDR1 RASQGINSFLA (SEQ ID NO: 17), CDR2 AADSIQS (SEQ ID NO: 18), and CDR3 QKSVYFPPT (SEQ ID NO: 16); or (iv) having heavy chain CDR sequences of CDR1 GSLSGYPWS (SEQ ID NO: 11), CDR2 ETDVSGWTDYNPSLKS (SEQ ID NO: 12), and CDR3 ARDGRRMGTPSFDI (SEQ ID NO: 13) and light chain CDR sequences of CDR1 RASKGISSFLA (SEQ ID NO: 20), CDR2 AASSLNS (SEQ ID NO: 15), and CDR3 QKAVYFPPT (SEQ ID NO: 19); or (v) having heavy chain CDR sequences of CDR1 GSLSGYPWS (SEQ ID NO: 11), CDR2 ETDVSGWTDYNPSLKS (SEQ ID NO: 12), and CDR3 ARDGRRMGTPSFDI (SEQ ID NO: 13) and light chain CDR sequences of CDR1 RASQGISSFLA (SEQ ID NO: 21), CDR2 AASSLQS (SEQ ID NO: 22), and CDR3 QSAVYFPPT (SEQ ID NO: 23). The antibody or antibody fragment may be encoded by variable sequences having:

(a) a heavy chain nucleotide sequence having at least or about 70%, 80%, 90%, 95%, 96%, 97%, 98%, 99%, or 100% identity to

(SEQ ID NO: 24)
CAAGTTCAACTCCAACAATGGGGTGCCGGGCTGCTGAAGCCGTCCGAAAC
GTTGAGTCTCACCTGCGCCGTGTACGGCGGCAGTCTTTCAGGCTATCCAT
GGTCCTGGATAAGGCAGCCCCCCGGCAAGGGTCTCGAATGGATAGGTGAG
ACGGACGTGTCTGGCTGGACAGATTACAACCCTTCTCTGAAGTCTCGCGT
TACCATTTCCGTAGATACAAGTAAGAATCAGTTCTCACTGAAGCTCAGTT
CTGTAACCGCTGCCGACACCGCAGTGTACTATTGTGCAAGAGATGGACGC
AGGATGGGAACTCCAAGCTTTGACATTTGGGGCCAGGGTACGATGGTGAC
TGTTTCCTCCGCCTCAACGAAAGGGCCATCTGTATTCCCATTGGCTCCGA
GTAGTAAGAGTACAAGTGGTGGGACCGCAGCGCTGGGATGCCTCGTCAAG
GATTATTTTCCCGAACCAGTAACTGTAAGTTGGAATAGCGGGGCATTGAC
AAGCGGCGTTCACACCTTTCCAGCGGTGCTTCAATCTTCTGGACTCTACT
CATTGAGCTCAGTTGTCACCGTTCCTTCAAGTTCCTTGGGCACGCAGACA
TATATCTGCAATGTGAATCATAAACCTTCTAATACGAAGGTCGATAAGAA
AGTGGAGCCAAAATCATGTGATAAGACGCATACTTGCCCCCCCTGCCCAG
CCCCTGAGCTGTTGGCAGGTCCCGATGTCTTTCTCTTTCCGCCCAAGCCG
AAGGATACGCTTATGATCTCCAGGACTCCTGAGGTGACTTGCGTCGTTGT
CGACGTATCCCACGAAGATCCTGAGGTGAAGTTTAATTGGTATGTCGACG
GAGTAGAGGTGCATAATGCTAAAACGAAACCTCGGGAAGAGCAATATAAT
TCCACTTACAGAGTAGTGAGCGTTCTCACAGTTTTGCATCAGGACTGGCT
TAACGGAAAGGAGTATAAATGTAAGGTCAGTAACAAAGCTCTTCCAGCTC
CCGAGGAGAAAACAATTTCAAAGGCAAAGGGCCAACCTAGGGAACCGCAG
GTGTATACTCTCCCCCCAAGCCGCGAAGAAATGACGAAAAACCAGGTGTC
CCTCACTTGCCTGGTCAAAGGTTTCTATCCTTCTGACATAGCGGTGGAGT
GGGAGAGTAACGGCCAACCAGAGAACAATTATAAGACCACGCCTCCAGTT
CTGGATTCCGATGGATCATTCTTTCTGTACTCTAAATTGACGGTTGACAA
ATCCCGCTGGCAGCAGGGAAACGTATTTTCATGTTCCGTGATGCATGAAG
CTTTGCACAATCACTACACTCAAAAGAGTTTGAGCTTGTCACCGGGAAAG
TAA,

and

(b) a light chain nucleotide sequence having at least or about 70%, 80% 90%, 95%, 96%, 97%, 98%, 99%, or 100% identity to:

(SEQ ID NO: 25)
GACATTCAGATGACGCAAAGCCCCAGCTCTGTCTCAGCTTCCGTGGGTGA
TAGGGTGACTATTACATGTCGAGCCTCACAAGATATTAATAGCTTTCTTG
CATGGTATCAGCAAAAGCCAGGGAAGGCACCTAAGCTCCTGATTTATGCT
GCCTCTTCTTTGAATAGCGGGGTCCCCTCCCGCTTCTCAGGATCTGGGTC
AGGGACTGACTTCACGCTGACTATATCCTCACTCCAACCAGAAGATTTCG
CCACTTATTACTGCCAAAAATCCGTATATTTCCCGCCCACATTCGGTGGT
GGGACAAAAGTGGAAATCAAGAGAACTGTCGCTGCCCCATCTGTTTTCAT
CTTTCCACCGAGCGACGAACAGCTCAAAAGCGGCACTGCGAGTGTTGTTT
GTCTGCTGAATAACTTCTATCCCAGGGAAGCAAAGGTGCAGTGGAAGGTA
GACAATGCTCTGCAATCCGGGAATAGTCAGGAATCCGTCACGGAGCAAGA
CAGTAAGGACTCCACGTATTCCTTGAGTAGTACATTGACCCTCAGTAAAG
CGGATTACGAGAAACACAAAGTTTACGCTTGTGAAGTAACGCATCAGGGG
TTGTCCAGTCCGGTTACCAAATCCTTCAATCGGGGAGAGTGTTGA.

The antibody or antibody fragment may comprise a heavy chain with an amino acid sequence having at least or about 70%, 80% 90%, 95%, 96%, 97%, 98%, 99%, or 100% identity to:

(SEQ ID NO: 26)
QVQLQQWGAGLLKPSETLSLTCAVYGGSLSGYPWSWIRQPPGKGLEWIG
ETDVSGWTDYNPSLKSRVTISVDTSKNQFSLKLSSVTAADTAVYYCARD
GRRMGTPSFDIWGQGTMVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLAGPDVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPEEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK;

and a light chain with an amino acid sequence having at least or about 70%, 80% 90%, 95%, 96%, 97%, 98%, 99%, or 100% identity to

(SEQ ID NO: 27)
DIQMTQSPSSVSASVGDRVTITCRASQDINSFLAWYQQKPGKAPKLLIYA
ASSLNSGVPSRFSGSGSGTDFTLTISSLQPEDFATYYCQKSVYFPPTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

TABLE 4
CDR Heavy and Light Chain Sequences for Ab-38002

| | CDRH1 | CDRH2 | CDRH3 |
|---|---|---|---|
| Antibody | (SEQ ID NO:) | (SEQ ID NO:) | (SEQ ID NO:) |
| 38002 | GSLSGYPWS | ETDVSGWTDYNP | ARDGRRMGTPSFDI |
| Heavy | (SEQ ID | SLKS | (SEQ ID NO: 13) |
| BIPDL4-3₄ | NO: 11) | (SEQ ID | |
| | | NO: 12) | |
| 38002 | RASQDINSFLA | AASSLNS | QKSVYFPPT |
| Light | (SEQ ID | (SEQ ID | (SEQ ID NO: 16) |
| BiPDL4-3₄ | NO: 14) | NO: 15) | |

There is also provided a method of treating cancer in a subject comprising contacting a PD-L1-, PD-L2-, or PD-L1 and PDL2-positive cancer cell in a subject with an antibody as described above. The PD-L1-, PD-L2-, or PD-L1 and PDL2-positive cancer cell may be a solid tumor cell, such as a lung cancer cell, brain cancer cell, head and neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, esophageal cancer cell, a lymphoma cell, a renal cell carcinoma cell, or may be a leukemia or myeloma such as acute myeloid leukemia, chronic myelogenous leukemia or multiple myeloma.

Free STING agonists via intratumoral injection consistently see a response in injected tumor. Rely on T cells primed in the injected tumor to eliminate cancer at other sites of metastases.

The method may further comprise contacting the PD-L1-, PD-L2-, or PD-L1 and PDL2-positive cancer cell with a second anti-cancer agent or treatment, such as chemotherapy, radiotherapy, immunotherapy, hormonal therapy, or toxin therapy. The second anti-cancer agent or treatment may inhibit an intracellular PD-L1 or PD-L2 function. The second anticancer agent or treatment may be given at the same time as the first agent or given before and/or after the agent. The PD-L1 or PD-L2-positive cancer cell may be a metastatic cancer cell, a multiply drug resistant cancer cell or a recurrent cancer cell.

The antibody fragment may be a recombinant scFv (single chain fragment variable) antibody, a single domain antibody, Fab fragment, F (ab′) 2 fragment, or Fv fragment. The antibody may be a chimeric antibody, a humanized antibody, or an IgG. The antibody may be a human antibody, murine antibody, an IgG, a humanized antibody or a humanized IgG. The antibody or antibody fragment may further comprise a label, such as a peptide tag, an enzyme, a magnetic particle, a chromophore, a fluorescent molecule, a chemiluminescent molecule, or a dye. The antibody or antibody fragment may further comprise an antitumor drug linked thereto, such as linked to the antibody or antibody fragment through a photolabile linker or an enzymatically-cleaved linker. The antitumor drug may be a toxin, a radioisotope, a cytokine or an enzyme. The antibody or antibody fragment may be conjugated to a nanoparticle or a liposome.

In another embodiment, there is provided a method of treating a glioma in a subject comprising delivering to the subject an antibody or antibody fragment alone or in combination with a STING agonist of the invention, wherein the antibody or antibody fragment comprises (i) heavy chain CDR sequences of CDR1 SEQ ID NO:11, CDR2 SEQ ID NO:12, and CDR3 SEQ ID NO: 13; and light chain CDR sequences of CDR1 SEQ ID NO:14, CDR2 SEQ ID NO:15, and CDR3 SEQ ID NO:16; (ii) heavy chain CDR sequences of CDR1 SEQ ID NO:11, CDR2 SEQ ID NO: 12, and CDR3 SEQ ID NO:13 and light chain CDR sequences of CDR1 SEQ ID NO:17, CDR2 SEQ ID NO:18, and CDR3 SEQ ID NO:19; or (iii) having heavy chain CDR sequences of CDR1 SEQ ID NO:11, CDR2 SEQ ID NO:12, and CDR3 SEQ ID NO:13, and light chain CDR sequences of CDR1 SEQ ID NO:17, CDR2 SEQ ID NO:18, and CDR3 SEQ ID NO:19; or (iv) having heavy chain CDR sequences of CDR1 SEQ ID NO:11, CDR2 SEQ ID NO:12, and CDR3 SEQ ID NO:13 and light chain CDR sequences of CDR1 SEQ ID NO:20, CDR2 SEQ ID NO:21, and CDR3 SEQ ID NO:22; or (v) having heavy chain CDR sequences of CDR1 SEQ ID NO:11, CDR2 SEQ ID NO:12, and CDR3 SEQ ID NO:13 and light chain CDR sequences of CDR1 SEQ ID NO: 21, CDR2 SEQ ID NO:22, and CDR3 SEQ ID NO:23. The antibody fragment may be a recombinant scFv (single chain fragment variable) antibody, Fab fragment, F (ab′h fragment, or Fv fragment. The antibody may be an IgG. The antibody may be a chimeric antibody or a humanized antibody. Delivering may comprise antibody or antibody fragment administration, or genetic delivery with an RNA or DNA sequence or vector encoding the antibody or antibody fragment.

The antibody or antibody fragment may be encoded by light and heavy chain variable sequences as set forth in SEQ ID NO:24 and SEQ ID NO:25, or may be encoded by light and heavy chain variable sequences having at least or about 70%, 80%, 90%, 95%, 96%, 97%, 98%, 99%, or 100% identity to sequences from SEQ ID NO:24 and SEQ ID NO:25. The antibody or antibody fragment may comprise light and heavy chain variable sequences according to sequences from SEQ ID NO:26 and SEQ ID NO:27, may comprise light and heavy chain variable sequences having at least or about 70%, 80%, 90%, 95%, 96%, 97%, 98%, 99%, or 100% identity to sequences from SEQ ID NO:26 and SEQ ID NO:27, or may comprise light and heavy chain variable sequences having at least or about 95%, 96%, 97%, 98%, 99%, or 100% identity to sequences from SEQ ID NO:26 and SEQ ID NO:27.

Also provided is a monoclonal antibody, wherein the antibody or antibody fragment is characterized by (i) heavy chain CDR1 SEQ ID NO:11, CDR2 SEQ ID NO:12, and CDR3 SEQ ID NO: 13; and light chain CDR sequences of CDR1 SEQ ID NO:14, CDR2 SEQ ID NO:15, and CDR3 SEQ ID NO:16; (ii) heavy chain CDR sequences of CDR1 SEQ ID NO:11, CDR2 SEQ ID NO: 12, and CDR3 SEQ ID NO:13 and light chain CDR sequences of CDR1 SEQ ID NO:17, CDR2 SEQ ID NO:18, and CDR3 SEQ ID NO:19; or (iii) having heavy chain CDR sequences of CDR1 SEQ ID NO:11, CDR2 SEQ ID NO:12, and CDR3 SEQ ID NO:13, and light chain CDR sequences of CDR1 SEQ ID NO:17, CDR2 SEQ ID NO:18, and CDR3 SEQ ID NO:19; or (iv) having heavy chain CDR sequences of CDR1 SEQ ID NO:11, CDR2 SEQ ID NO:12, and CDR3 SEQ ID NO:13 and light chain CDR sequences of CDR1 SEQ ID NO:20, CDR2 SEQ ID NO:21, and CDR3 SEQ ID NO:22; or (v) having heavy chain CDR sequences of CDR1 SEQ ID NO:11, CDR2 SEQ ID NO:12, and CDR3 SEQ ID NO:13 and light chain CDR sequences of CDR1 SEQ ID NO: 21, CDR2 SEQ ID NO:22, and CDR3 SEQ ID NO:23. The antibody fragment may be a recombinant scFv (single chain fragment variable) antibody, Fab fragment, F (ab′) 2 fragment, or Fv fragment. The antibody may be a chimeric antibody, a humanized antibody, or an IgG.

Also provided is a nucleic acid encoding the antibody or antibody fragment having light and heavy chain variable sequences as set forth SEQ ID NO:24 and SEQ ID NO:25, or a nucleic acid encoding light and heavy chain variable sequences having at least or about 70%, 80%, 90%, 95%, 96%, 97%, 98%, 99%, or 100% identity to sequences from SEQ ID NO:24 and SEQ ID NO: 25. Also provided is a nucleic acid encoding the antibody or antibody fragment having light and heavy chain variable sequences according to sequences from SEQ ID NO:26 and SEQ ID NO: 27, or a nucleic acid encoding an antibody or an antibody fragment comprising light and heavy chain variable sequences having at least or about 70%, 80%, 90%, 95%, 96%, 97%, 98%, 99%, or 100% identity to sequences from SEQ ID NO:26 and SEQ ID NO:27.

A further embodiment comprises a cancer vaccine comprising one or more antibodies or antibody fragments characterized by heavy and light chain CDR sequences as described above. At least one antibody fragment may be a recombinant scFv (single chain fragment variable) antibody, a single domain antibody, Fab fragment, F (ab′) 2 fragment, or Fv fragment. At least one of antibody may be a chimeric antibody, or an IgG. At least one antibody or antibody fragment may be encoded by light and heavy chain variable sequences as set forth herein, may be encoded by light and heavy chain variable sequences as described above, and may be encoded by light and heavy chain variable sequences having at least or about 70%, 80%, 90%, 95%, 96%, 97%, 98% or 99% identity to sequences as described above. At least one antibody or antibody fragment may comprise light and heavy chain variable sequences according to sequences as described above and may comprise light and heavy chain variable sequences having at least or about 70%, 80%, 90%, 95%, 96%, 97%, 98% or 99% identity to sequences as described above.

In another embodiment there is provided a method of detecting PD-L1 or PD-L2 expressing cells in a subject comprising contacting a sample from said subject with an antibody or antibody fragment characterized by heavy and light chain CDR sequences as described above and detecting a PD-L1 or PD-L2 expressing cell in said sample by binding said antibody or antibody fragment to a cell in said sample. The sample may be a body fluid or a tissue sample. The cell may be a cancer cell, such as a lymphoma cell, breast cancer cell, or renal cell carcinoma cell. The cell may be a cell associated with immune suppression. The cell associated with immune suppression may be a non-cancerous cell in a tumor microenvironment, such as a stromal cell or endothelial cell. Detection may comprise ELISA, RIA, or Western blot. The method may further comprise performing the method a second time and determining a change in antigen levels as compared to the first assay. The antibody or antibody fragment may be encoded by light and heavy chain variable sequences as described above and may be encoded by light and heavy chain variable sequences having at least or about 70%, 80%, 90%, 95%, 96%, 97%, 98% or 99% identity to sequences as described above. The antibody or antibody fragment may comprise light and heavy chain variable sequences according to sequences described above and may comprise light and heavy chain variable sequences having at least or about 70%, 80%, 90%, 95%, 96%, 97%, 98% or 99% identity to sequences described above.

There is also provided a method of treating cancer in a subject comprising contacting a PD-L1- or PD-L2-positive cancer cell in a subject with an antibody as described above. The PD-L1- or PD-L2-positive cancer cell may be a solid tumor cell, such as a lung cancer cell, brain cancer cell, glioblastoma, head & neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, esophageal cancer cell, a lymphoma cell, a renal cell carcinoma cell, or may be a leukemia or myeloma such as acute myeloid leukemia, chronic myelogenous leukemia or multiple

### III. Immunodetection Methods

In still further embodiments, there are immunodetection methods for binding, purifying, removing, quantifying and otherwise generally detecting PD-L1 or PD-L2 and their associated antigens. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of PD-L1 and PD-L2 antibodies also is provided. The steps of various useful immunodetection methods have been described in the scientific literature, such as, e.g., Doolittle and BenZeev (1999), Gulbis and Galand (1993), De Jager et al. (1993), and Nakamura et al. (1987). In general, the immunobinding methods include obtaining a sample and contacting the sample with a first antibody in accordance with embodiments discussed herein, as the case may be, under conditions effective to allow the formation of immunocomplexes.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to PD-L1 and PD-L2 present. After this time, the sample-antibody composition, such as a tissue section, ELISA plate, dot blot or Western blot, will generally be washed to remove any nonspecifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAS

Immunoassays, in their most simple sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing PD-L1 and/or PD-L2 is added to the wells. After binding and washing to remove non-specifically bound immune complexes, the bound antigen may be detected. Detection may be achieved by the addition of another bispecific antibody directed to PD-L1 and PD-L2 linked to a detectable label, or by an anti-PD-L1 or anti-PD-L2 antibody that is linked to a detectable label. This type of ELISA is a simple “sandwich ELISA.” Detection may also be achieved by the addition of a second bispecific antibody to PD-L1 and PD-2, or an anti-PD-L1 or anti-PD-L2 antibody, followed by the addition of a third antibody that has binding affinity for the second antibody, with the third antibody being linked to a detectable label.

In another exemplary ELISA, the samples suspected of containing the PD-L1 and/or PD-L2 antigen are immobilized onto the well surface and then contacted with a bispecific anti-PD-L1 and anti-PD-L2 antibody. After binding and washing to remove non-specifically bound immune complexes, the bound bispecific anti-PD-L1 and anti-PD-L2 antibodies are detected. Where the initial bispecific anti-PD-L1 and anti-PDL2 antibodies are linked to a detectable label, the immune complexes may be detected directly. Again, the immune complexes may be detected using a second antibody that has binding affinity for the first bispecific anti-PD-L1 and anti-PD-L2 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to about 2 to 4 hours, at temperatures preferably on the order of 25° C. to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethylbenzthiazoline-6-sulfonic acid (ABTS), or H202, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/nondenaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pl), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probing. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fe region of the primary antibodies.

### C. Immunohistochemistry

The antibodies may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, there are immunodetection kits for use with the immunodetection methods described above. The immunodetection kits will thus comprise, in suitable container means, a first bispecific antibody that binds to PD-L1 and/or PD-L2 antigens, and optionally an immunodetection reagent.

In certain embodiments, the bispecific antibody to PD-L1 and PD-L2 may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with embodiments discussed herein.

The kits may further comprise a suitably aliquoted composition of the PD-L1 and PD-L2 antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits will also include a means for containing the antibody, antigen, and any other reagent containers in close confinement for commercial sale. Such containers may include injection or blow-molded plastic containers into which the desired vials are retained.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1-Intratumoral Delivery of STING Agonist Results in Radiographic Response in Spontaneous Canine High-Grade Glioma

A single ˜5 mm-diameter burr hole craniectomy was made at entry point determined by Brainsight Neuronavigation System.

A 100 uL-Hamilton syringe secured in a calibrated holder on an x-y stage containing 50 μL of Compound C at varying concentrations

Contrast enhancing target was selected using the MRI reconstruction.

Compound of Formula C was injected at a rate of 2 uL/minute.

Compound of Formula C was administered via repeated intratumoral injections up to 20 μg in 50 uL in a large animals (canines) with spontaneous unresected gliomas.

The MTD of STING agonist Compound C is in the range of 15-20 μg. A MTD will need to be re-defined in human subjects in which standard-of-care tumor debulking is performed prior to the intratumoral administration of a STING agonist.

A single injection of Compound of Formula C can produce radiographic responses in spontaneously arising high-grade gliomas in the dog.

Compound of Formula C induced an early inflammatory response characterized by mixed infiltrate, edema, and necrosis which could be modulated with interventions such as ventriculostomy and/or medical strategies not currently available for canines.

### Example 2

The Compound of Formula A was analyzed in vitro for potency in activating the human (THP-1 reporter cells) and mouse (293 reporter cells) STING pathways. Antibody Ab-27907 was tested in vitro for potency in reporter cell assays measuring PD-1 pathway blockade, antibody-dependent cellular cytotoxicity (ADCC), and antibody-dependent cellular phagocytosis (ADCP). The Compound of Formula A was tested in vivo, alone and in combination with Ab-27907, against mouse models of melanoma (B16F10 expressing mouse PD-L2) and mammary adenocarcinoma (TS/A). Mice with established tumors were treated with the Compound of Formula A intratumorally at 10 μg/dose twice (days 11 and 14 for B16F10-PDL2 and days 23 and 26 for TS/A) and with Ab-27907 at 10 mg/kg twice a week for 3 weeks, starting with the first Formula A treatment. TS/A tumors were removed 32 days post-tumor challenge and analyzed via H&E, IHC and FACS to assess overall necrosis, T cell infiltration, and macrophage content.

### Results

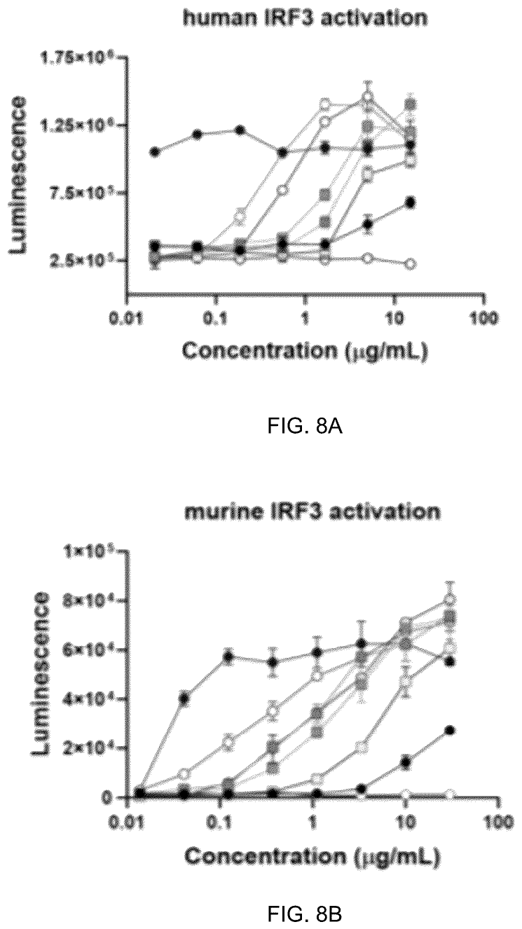

The potency of the Compound of Formula A was measured in an IRF3 reporter cell system and compared to that of a variety of other STING agonists (FIG. 8). The Compound of Formula A more effectively activates human and mouse STING relative to clinical benchmarks. It's EC₅₀ of 0.19 μg/ml and 0.28 μg/ml in mouse and human cell lines, respectively, was 12-175X lower than the other compounds.

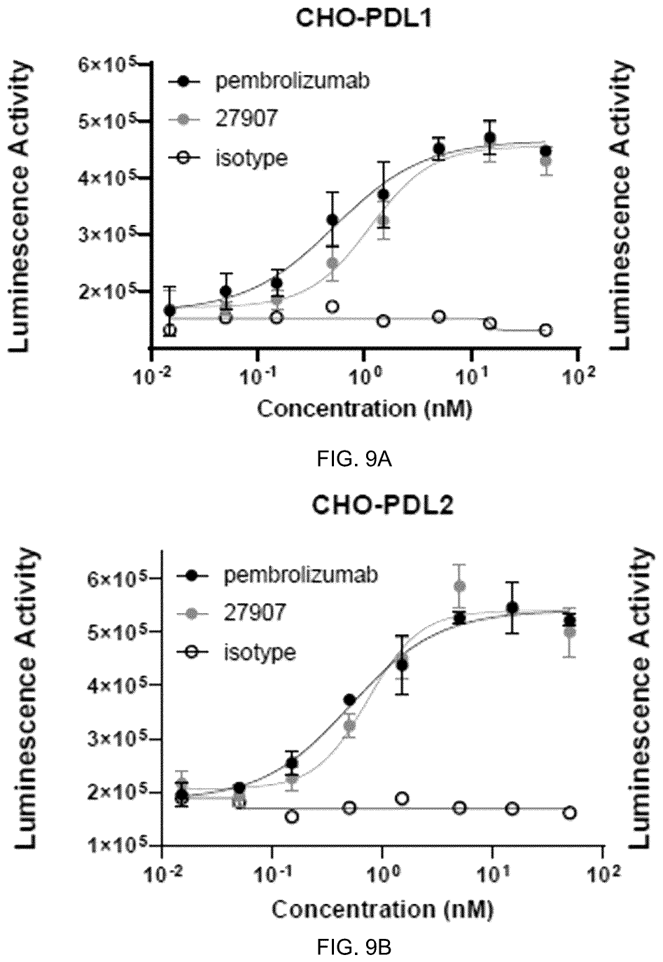

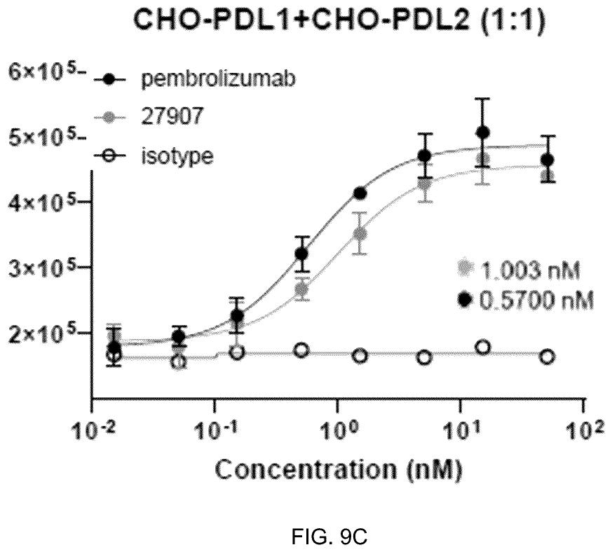

The ability of Ab-27907 to interrupt the PD-1 pathway through binding of the ligands PD-L1 and PD-L2 was tested in a reporter cell system. In this assay, PD-1 expressing effector cells are incubated with artificial antigen presenting cells expressing PD-L1 or PD-L2 with a T cell receptor (TCR). Without any antibodies, luminescence activity is low, as PD-1 binds its ligands, preventing TCR engagement and production of luciferase. As shown in FIG. 9, Ab-27907 blocks PD-1: PD-L1 as well as pembrolizumab and additionally blocks PD-1: PD-L2

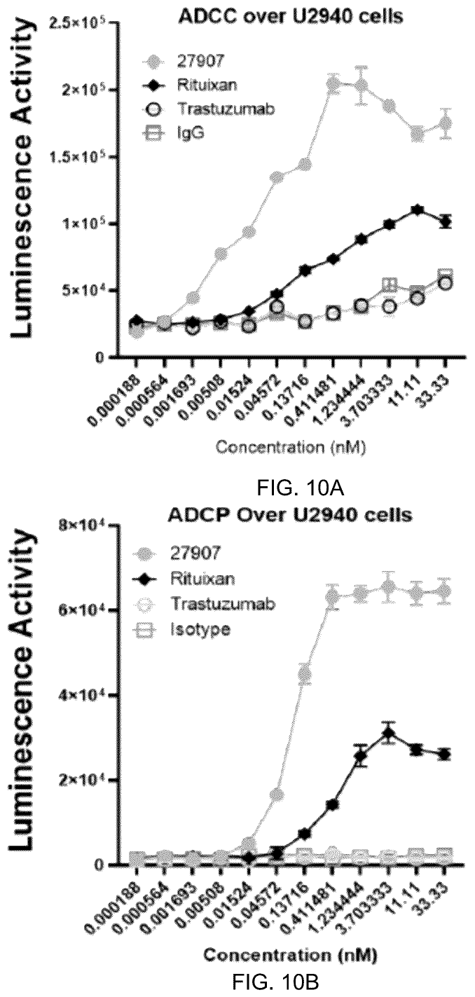

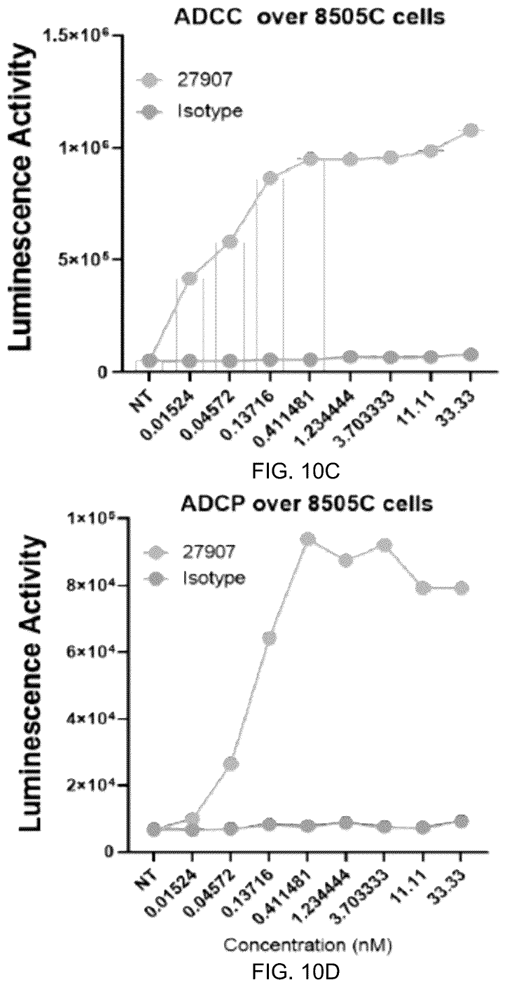

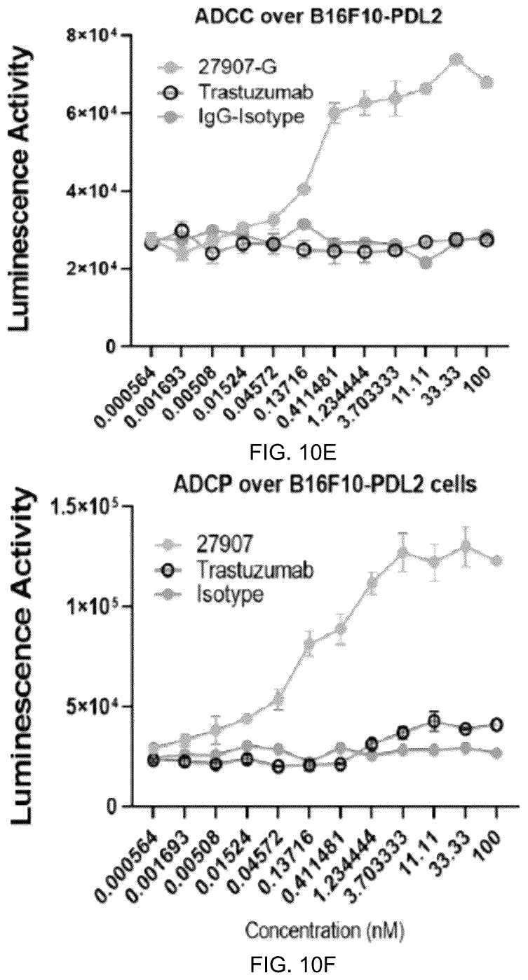

The capacity for Ab-27907 to induce ADCC and ADCP was tested in a reporter cell assay system and showed robust activity (FIG. 10).

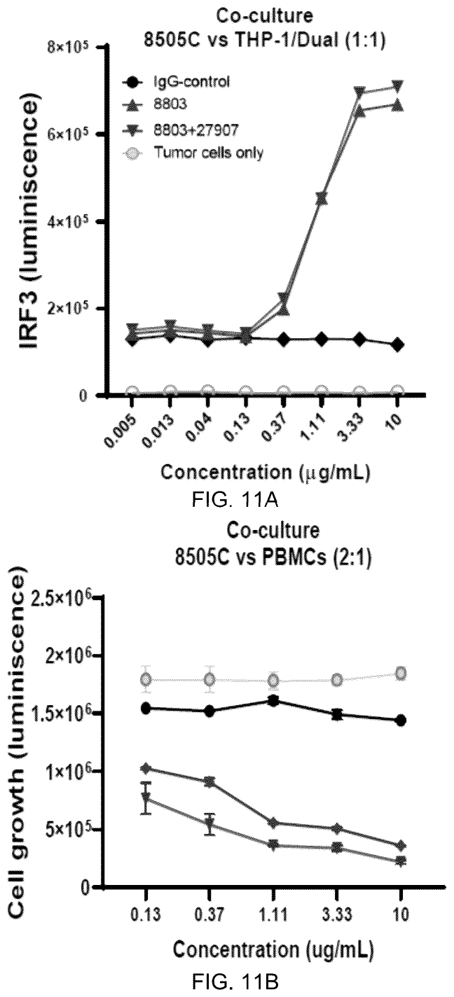

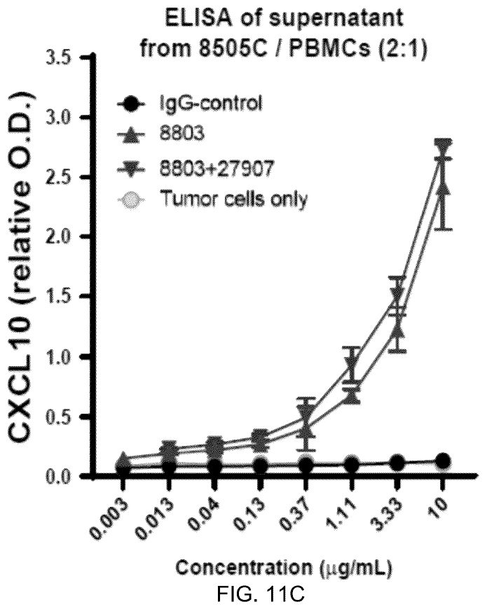

Compound 8803 (i.e., the Compound of Formula A) provoked a clear dose dependent response from THP-1 reporter cells, in inhibiting the growth of tumor cells in culture with PBMCs, and in inducing secretion of CXCL10. (FIG. 11).

### Example 3

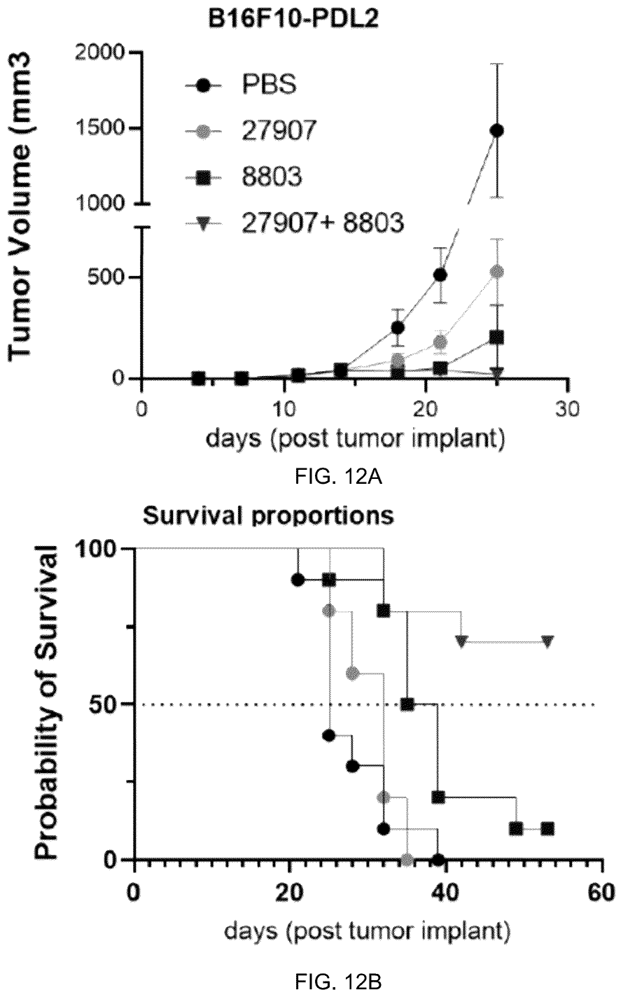

In vivo, the combination of Compound 8803 (i.e., Compound of Formula A) with Ab-27907 resulted in 70% overall survival in B16F10-PDL2, compared to control groups (FIG. 12). It is important to note that the size of tumors at the start of therapy were over 150 mm³ (˜11 days post tumor injection) thus limiting the activity of 27907 as monotherapy. Mice started dying in less than 21 days and IO therapies needs time to eliminate/remodel tumor and/or TME. Nevertheless, Ab-27907 significantly inhibited tumor growth for the first 2 weeks.

### Example 4

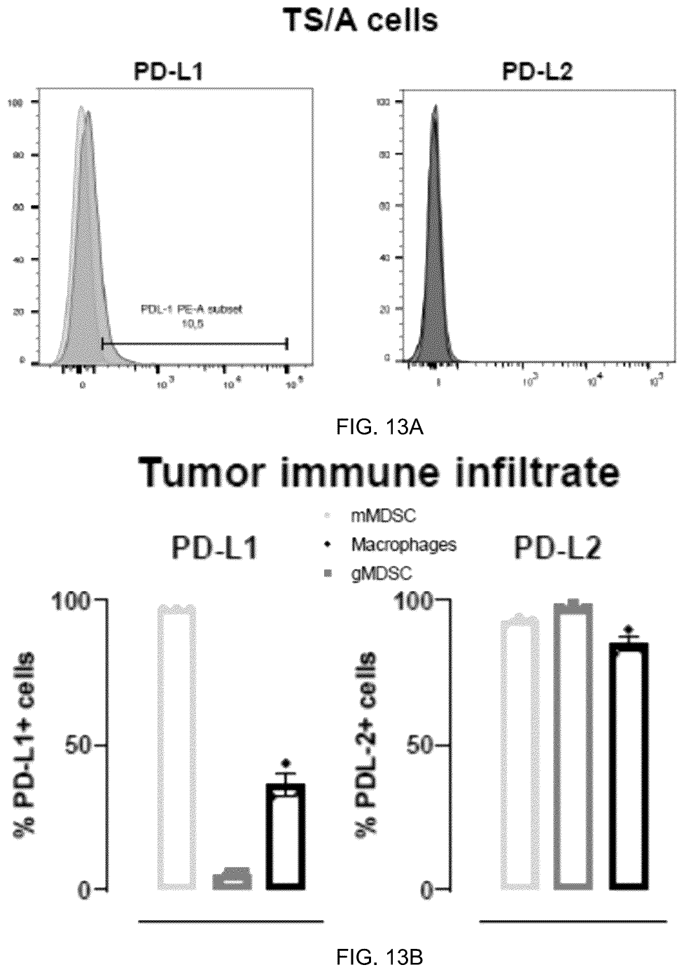

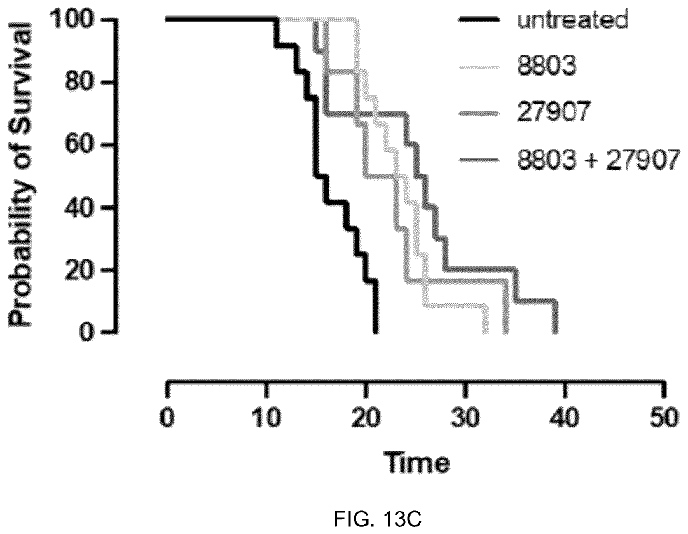

Combination treatment with Compound 8803 and Ab-27907 in the TS/A mouse model of mammary carcinoma inhibited tumor growth and, more importantly, extended overall survival. TS/A was chosen for this study in order to test efficacy of the combination treatment against tumors that have no PD-L2 expression and very limited (<10%) PD-L1 expression (FIG. 13).

As shown in FIG. 13, flow cytometry confirmed the very low expression of the PD-1 ligands on TS/A tumor cells. In contrast, analysis revealed strong expression of PD-L2 across myeloid cells within the TS/A tumor microenvironment. Infiltrating mMDSCs expressed high levels of PD-L1, while gMDSCs expressed less, and macrophages did not express PD-L1. This data supports the hypothesis that Ab-27907 is able to target PD-L1/PD-L2 expression on immune-suppressive cells throughout the tumor.

The combination treatment increased survival past 40 days, compared to 20 days in the untreated control group. Compound 8803 and Ab-27907 monotherapy also showed a significant extended survival versus control group.

### Example 5

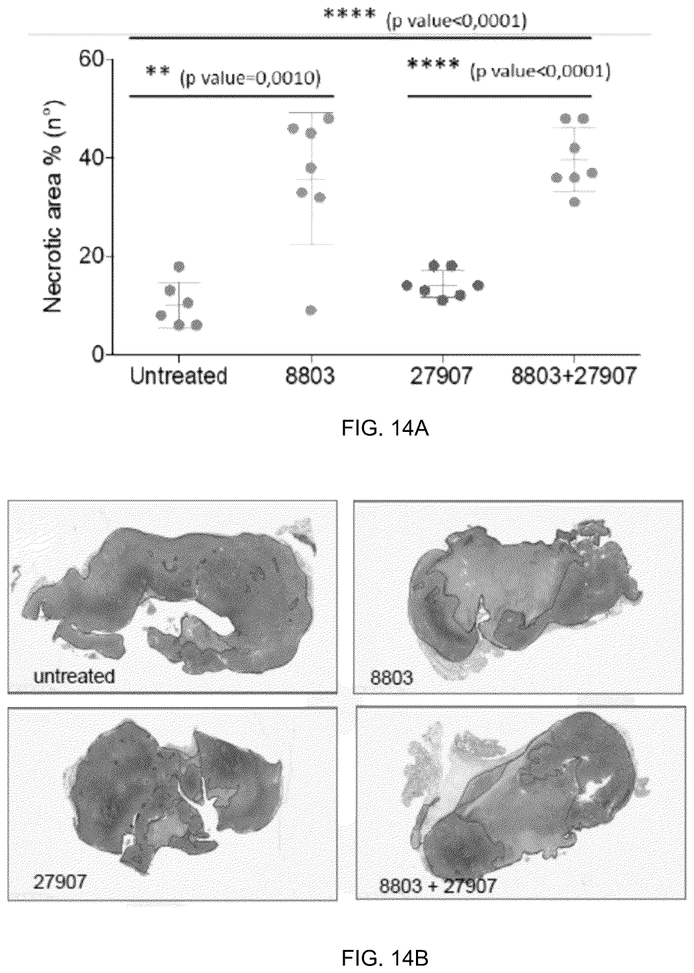

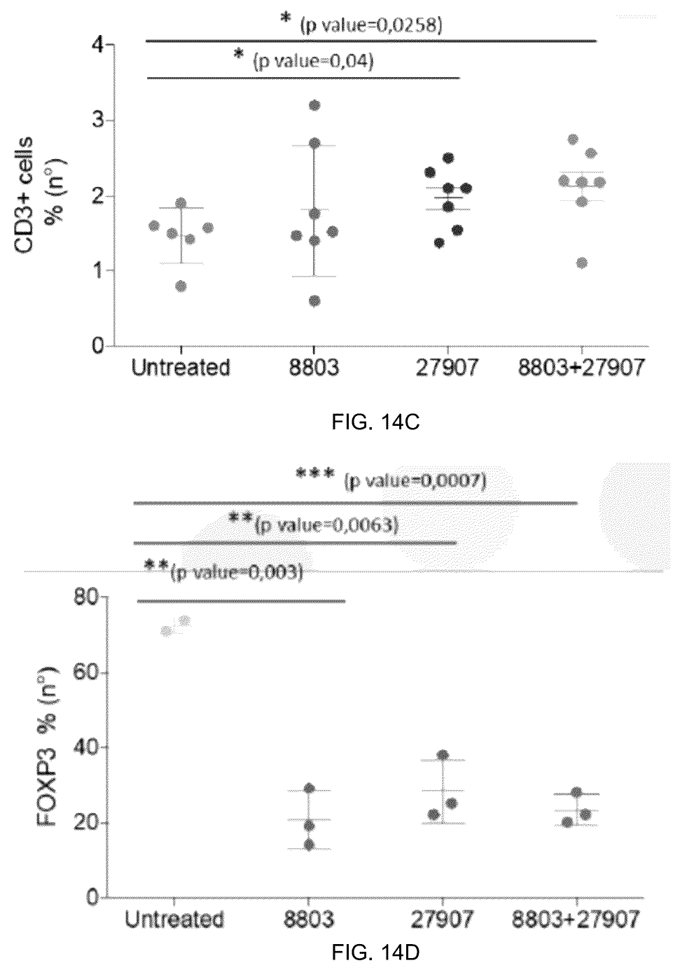

In the TS/A model, the combination therapy resulted in a large necrotic area compared to the respective control and individual treatments. CD3+ cells were more numerous in the tumors treated with the combination, while FoxP3+ cells showed a decrease in comparison with the controls (FIG. 14).

### Example 6

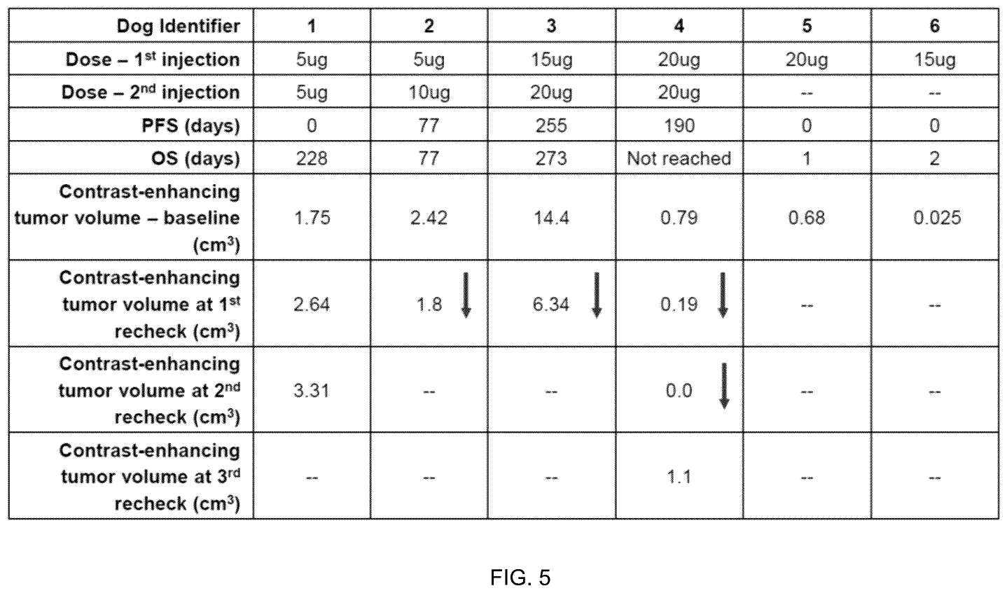

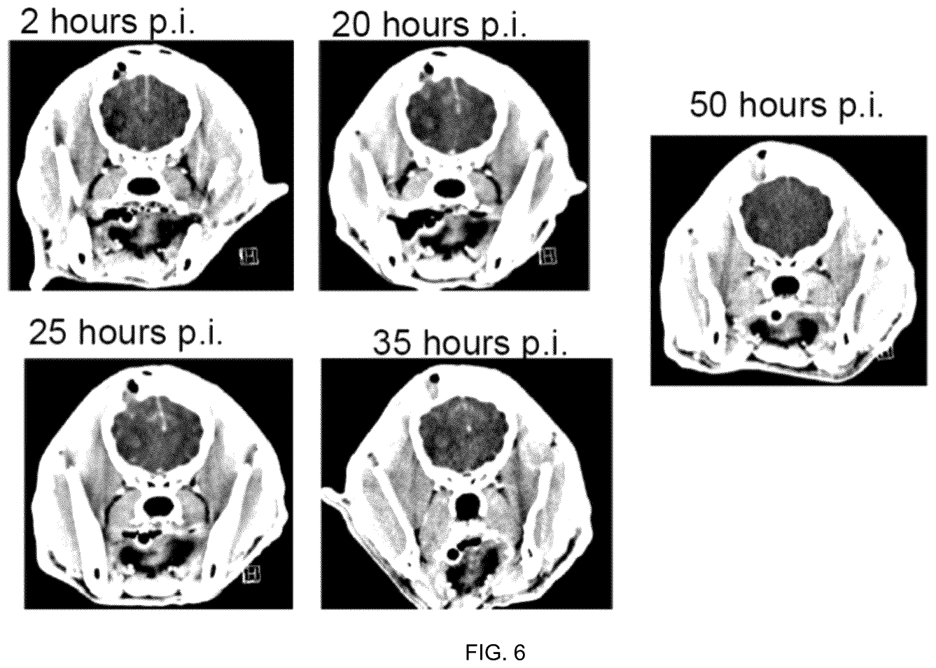

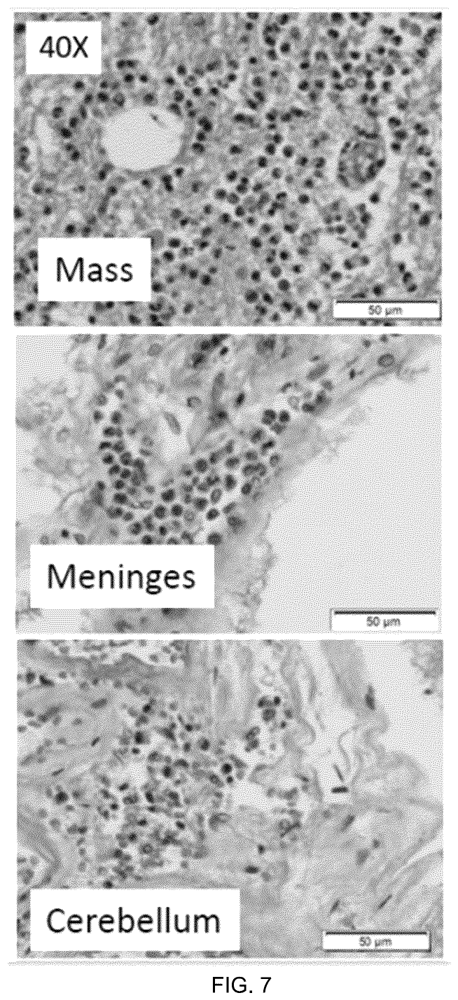

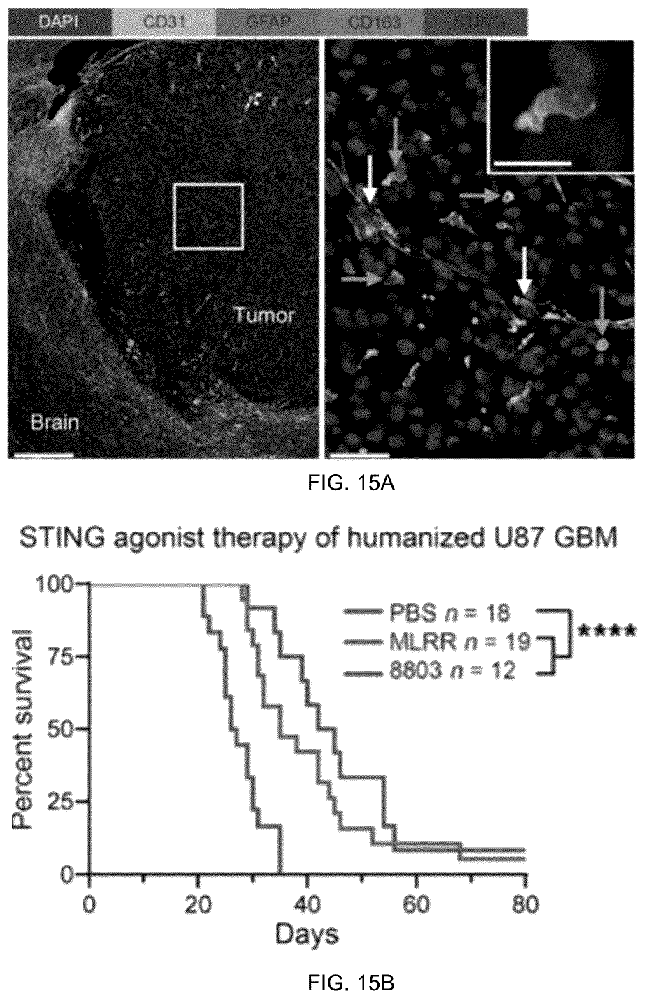

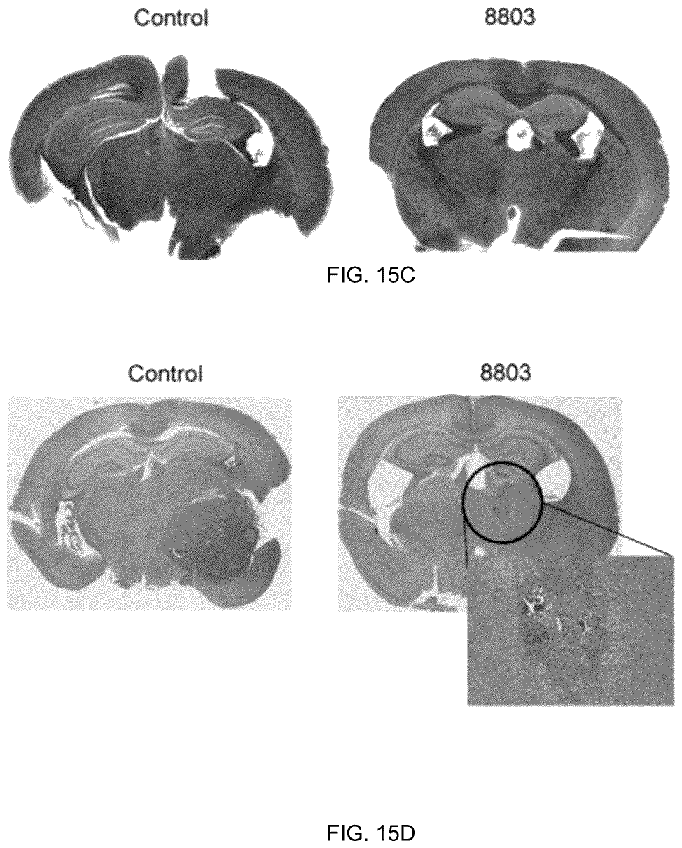

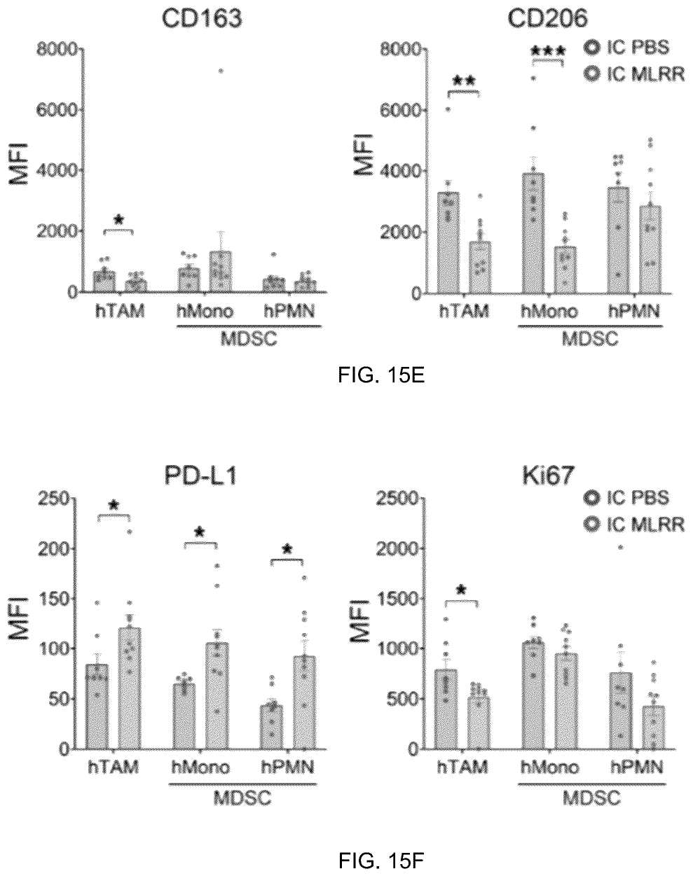

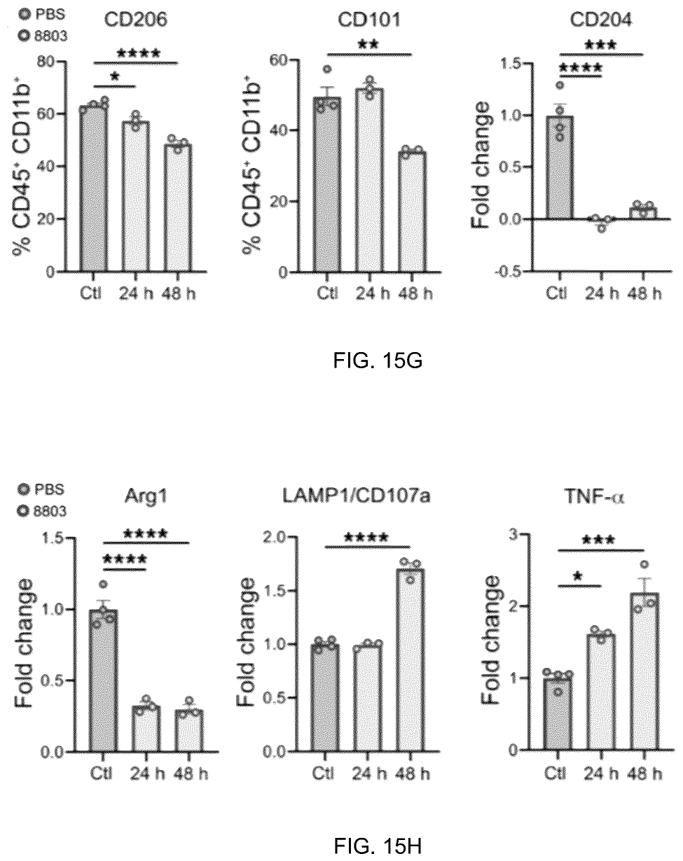

STING expression on human and murine glioma cells was analyzed (FIG. 15A). The cell lines U87, GL261, and CT-2A were selected for this analysis because they may recapitulate the human glioblastoma biology of epigenetic silencing of STING. Although there are significant genetic and immunologic similarities between the QPP glioblastoma lines and human glioblastoma, we sought to evaluate the effects of STING agonist therapy in CD34+ stem cell-humanized huNOG-EXL mice. Effectively, in the U87 tumors implanted in the humanized mice, STING expression at the protein level was observed on the endothelium (CD31+, white arrows) and within the CD163+ macrophages (green arrows) within the TME (FIG. 5A). Both 8803 and another prototype synthetic STING agonist, MLRR-S2-CDA, significantly extended survival of U87 tumor-bearing animals (both P<0.0001), confirming the efficacy of this approach in a system with human lymphocytes and myeloid cells (FIG. 15B). To evaluate whether this treatment induces an inflammatory response directed against the CNS, the mice were evaluated daily. No neurological symptoms were noted. When the mice succumbed to the tumor, the neuroaxis was stained for myelin with Luxol Fast Blue, and hematoxylin and eosin. On histologic examination by a board-certified neuropathologist, there was no evidence of autoimmune demyelinating encephalomyelitis (FIGS. 15, C and D). Additionally, neither perivascular lymphocytic infiltrates nor vascular fibrosis were seen, which otherwise might have suggested induction of low-grade autoimmunity. To verify that the absence of induced autoimmunity was not simply a function of the model analyzed, both C57BL/6J control and 8803-treated mice were analyzed similarly and likewise showed no evidence of demyelination. An invasion assay was conducted on glioma tumor cells, indicating that Compound 8803 (i.e., Compound of Formula A) does not enhance the underlying invasive properties of these cells. Cumulatively, these data indicate that the Compound of Formula A is exerting an immunological and therapeutic effect in a glioma model in which STING has been deactivated in the tumor cell. Although the STING-treated mice succumbed to the disease, the tumors were smaller than those of controls (FIG. 15D).

### Example 7

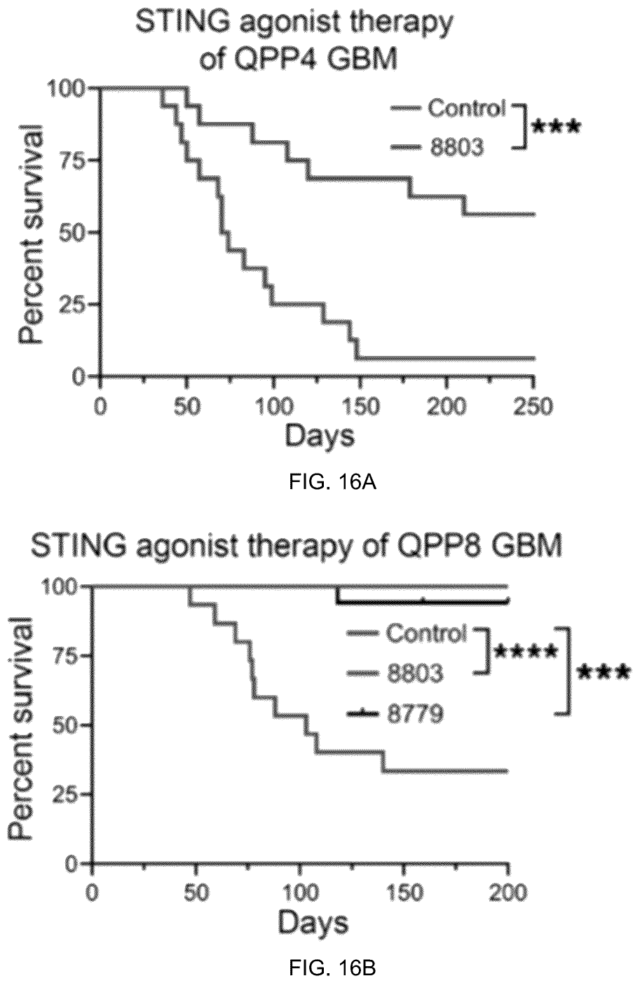

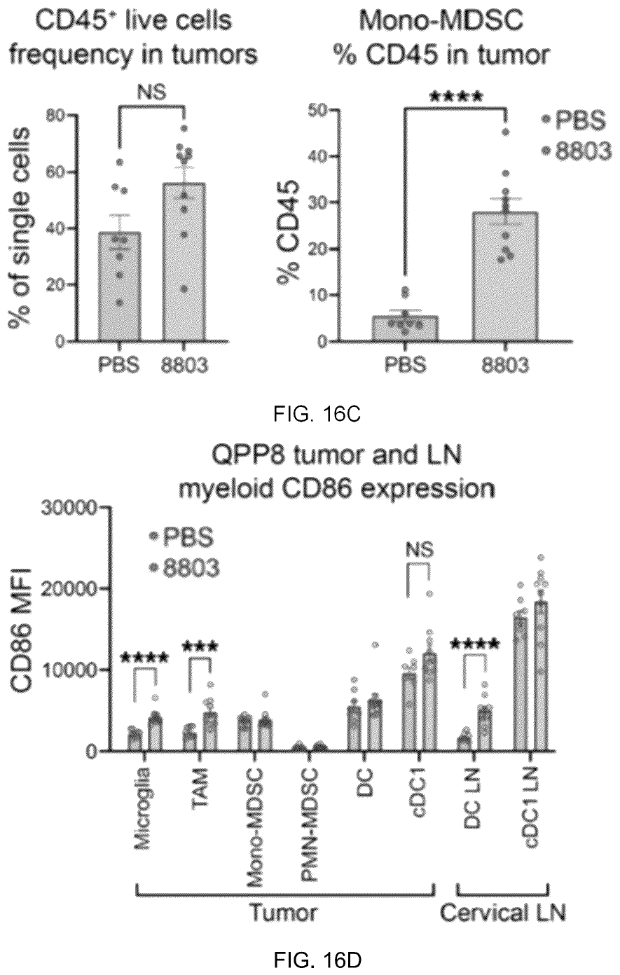

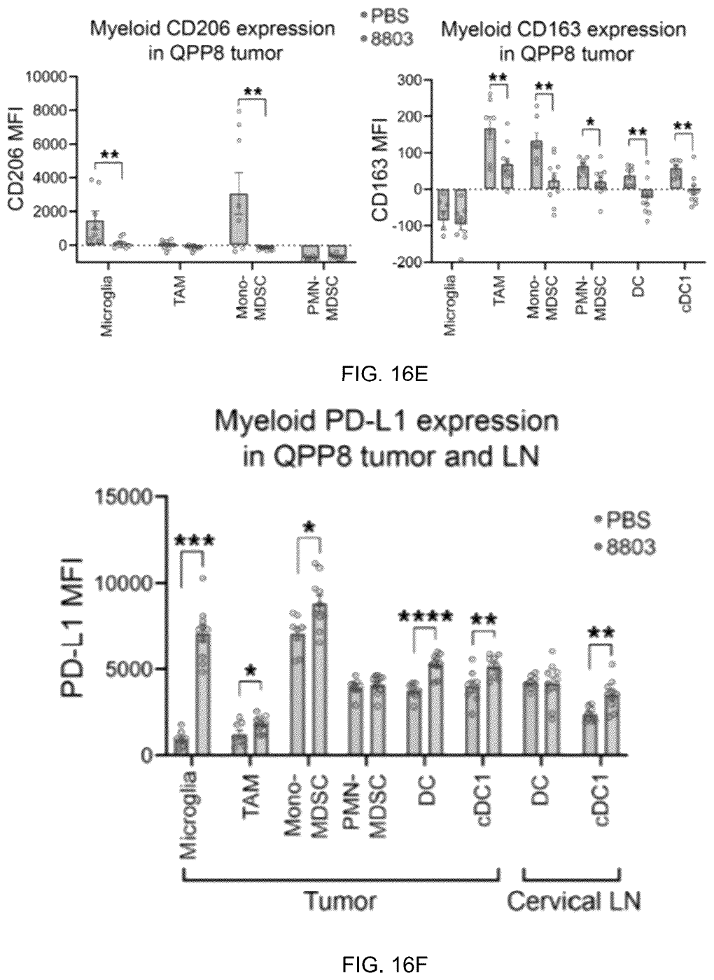

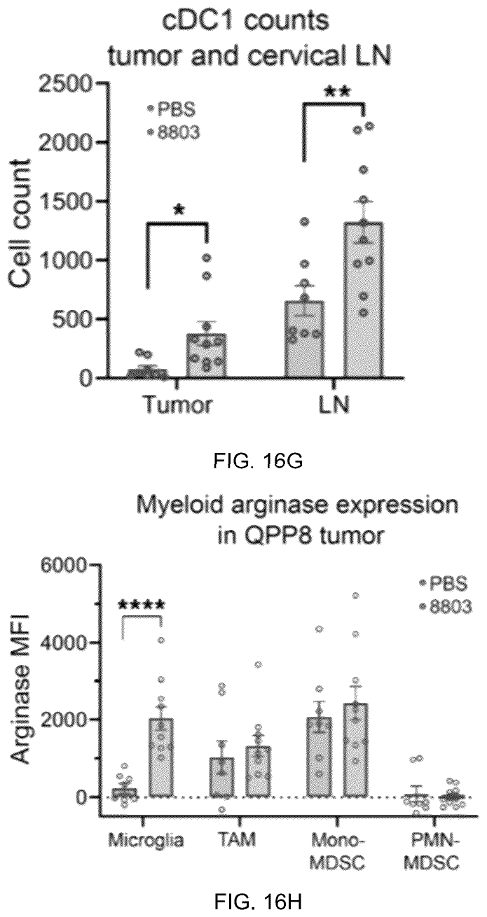

STING agonist Compound A increases myeloid activation in immune checkpoint blockade-refractory glioblastoma. QPP8 tumors were treated twice, 7 days apart, with 5 μg of the STING agonist Compound A and then isolated and analyzed by flow cytometry 48 hours following the second treatment. With STING agonist therapy, CD45+ immune cell frequency increased in the tumors primarily driven by the influx of CD11b+Ly6C+MDSCs (FIG. 16C). Costimulatory CD86 expression increased in the microglia, tumor-associated macrophages (TAMs), and the DC compartments in the tumor and the DCs residing in the cervical lymph nodes (LNs) (FIG. 16D). Across the glioblastoma myeloid stroma, we also observed decreased expression of the immunosuppressive markers CD206 and CD163 (FIG. 16E). Consistent with the increased IFN release associated with STING agonist therapy, expression of the immune checkpoint ligand PD-L1 also increased across the QPP8 myeloid stroma (FIG. 16F). Professional antigen cross-presenting conventional type 1 DCs (cDC1s) increased in frequency, both within the tumor and in the LNs (FIG. 16G). Paradoxically, arginase 1 (Arg1) expression, which is upregulated by STAT3 (30), increased across immune lineages in response to STING therapy (FIG. 16H).

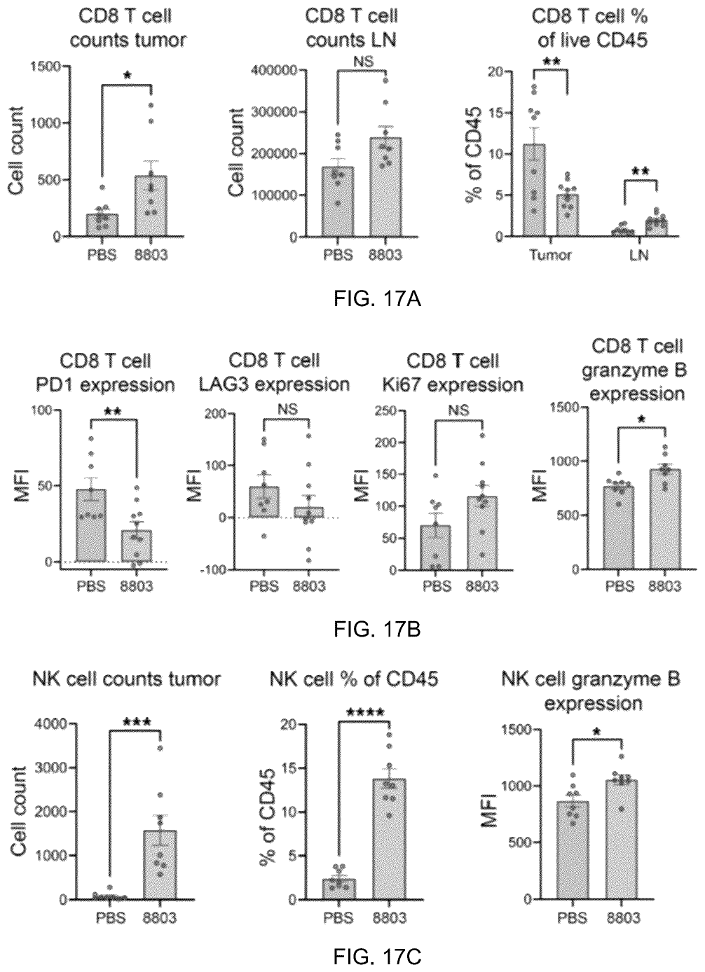

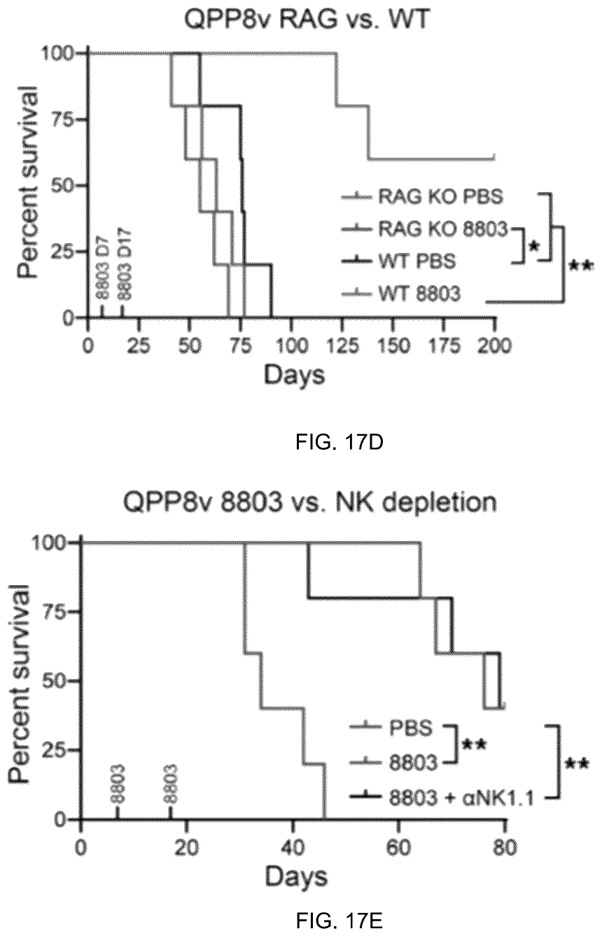

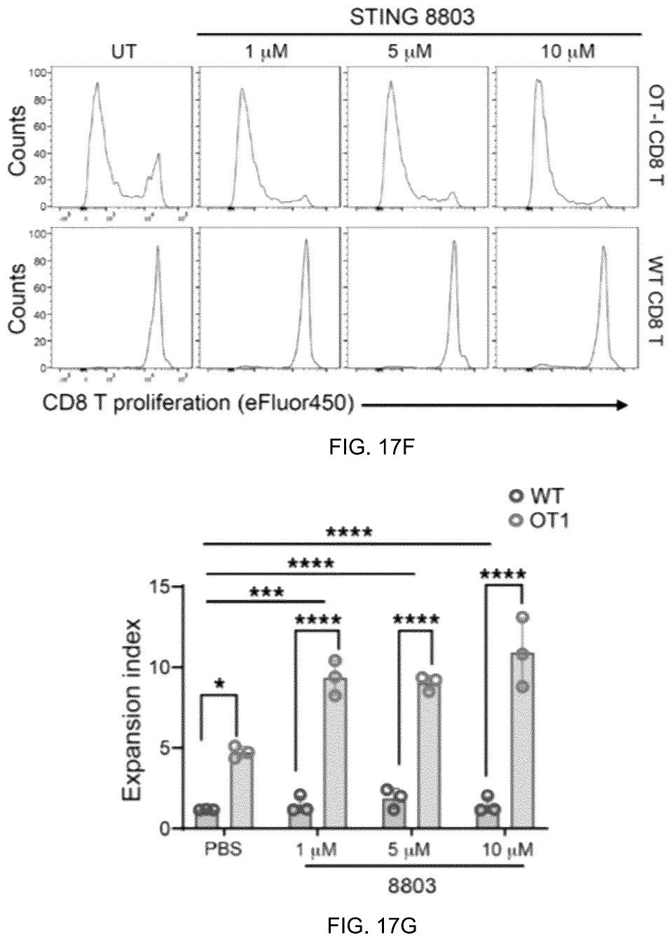

STING agonist Compound A functionally enhances both CD8+T and NK cell responses in glioblastoma. The number of intratumoral CD8+ T cells increased with 8803 treatments, but their frequency within the CD45+ population declined due to the higher proportional levels of myeloid expansion. In the cervical LNs, a higher CD8+ T cell proportion with STING agonist therapy could be observed. CD8+ T cells were less exhausted, with lower PD-1 and LAG-3 expression, had increased cytotoxic potential, and trended toward increased proliferation (FIG. 17B). NK cells increased in both number and as a fraction of the glioblastoma immune infiltrates with cytotoxic potential. To clarify the immune cell population mediating the effector response of Compound A, hereafter the QPP8v subclone was implanted into RAG−/−mice, which lack mature B and T cells (see Methods), and responses compared relative to WT C57BL/6J mice (FIG. 17D). The absence of T cells in the RAG−/−mice abolished the therapeutic effect of Compound A in comparison with the WT mice (FIG. 17D). Additionally, in vivo NK1.1 cell depletion did not ablate the therapeutic activity of 8803 (FIG. 17E), indicating that the immune effector population is the T cell. Finally, to ascertain whether Compound A can induce antigen-specific responses, GL261 cells expressing ovalbumin were pretreated with Compound A at 3 doses (1, 5, and 10 uM) and then cocultured with ovalbumin-specific TCR-transgenic OT-1 CD8+ T cells or WT control T cells (FIGS. 17F, 17G). Antigen-specific T cell expansion was detected at 1 μM and was not dose-dependent.

### Example 8

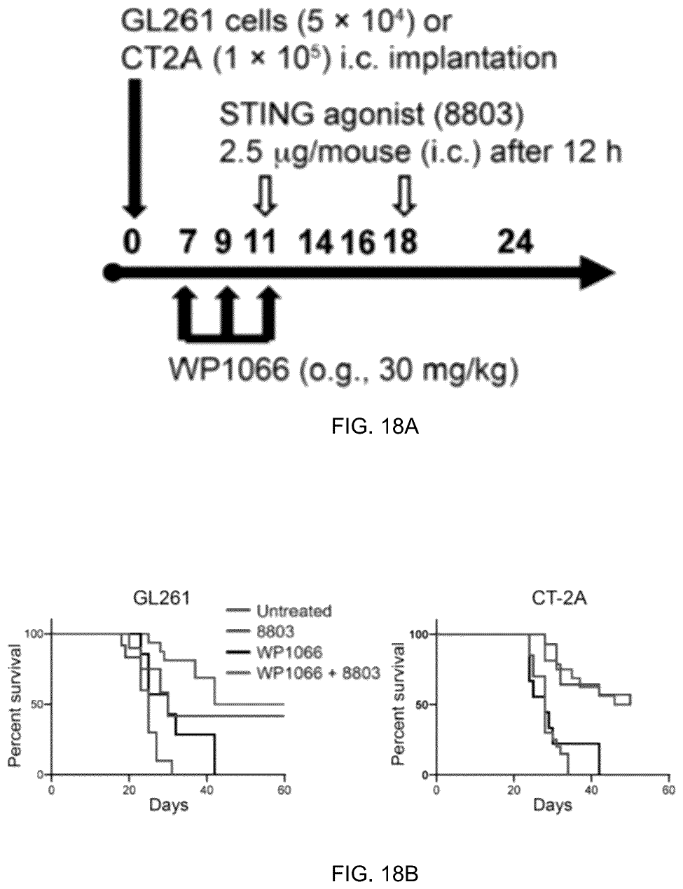

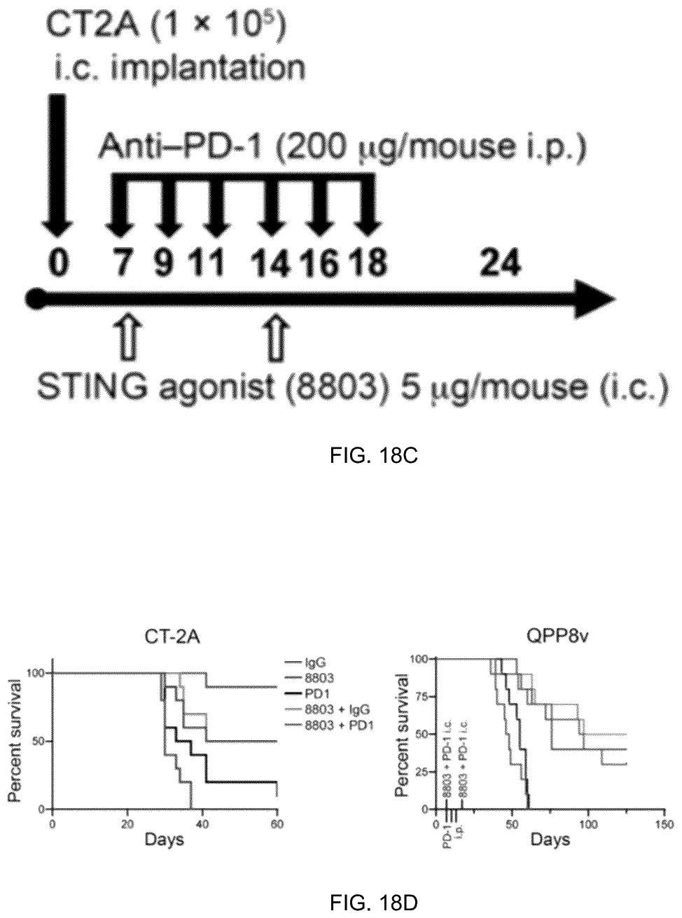

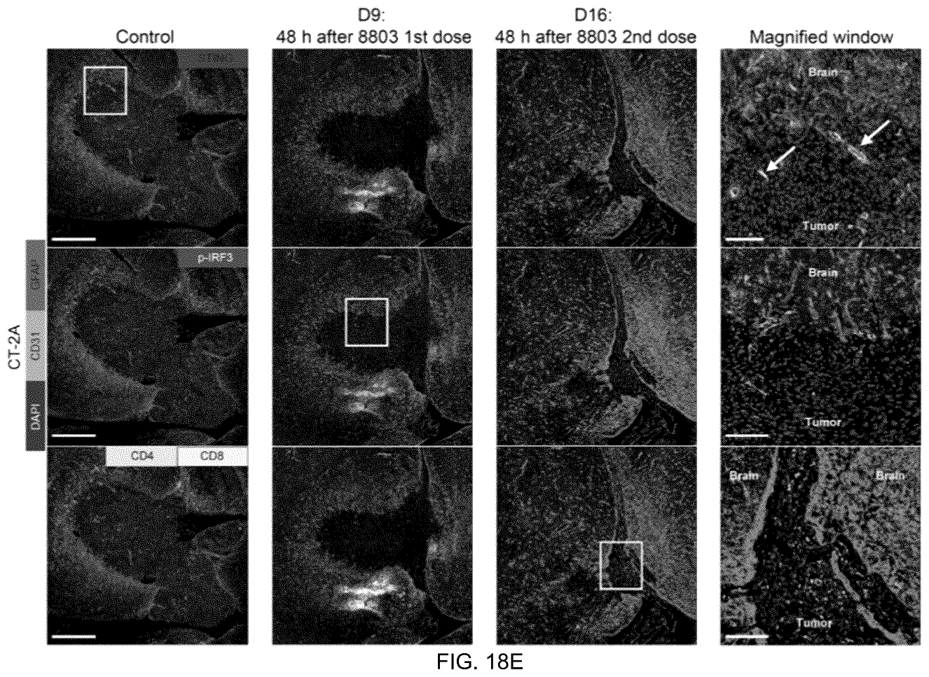

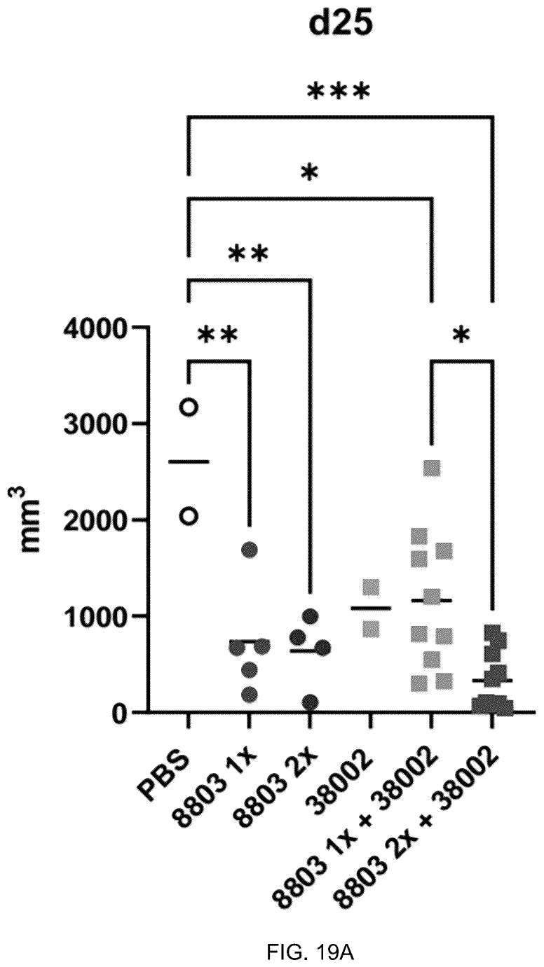

STING agonist 8803 (Compound A) demonstrates synergy with anti-PD-1. PD-1 is expressed on macrophages in the glioma microenvironment, and anti-PD-1 antibodies can induce proinflammatory M1 responses. Since a STING agonist would stimulate a pro-inflammatory response, the modulation of the PD-1 pathway may further potentiate the therapeutic effect of STING. As such, we evaluated synergy in the CT-2A and QPP8v immunocompetent murine glioma models. C57BL/6J mice bearing i.e. CT-2A or QPP8v tumors were treated with the STING agonist 8803 and/or anti-PD-1. The CT-2A mice were randomized to receive (i) IgG control (200 μg/mouse administered i.p. 3 times per week for 2 weeks); (ii) anti-PD-1 (200 μg/mouse administered i.p. 3 times per week for 2 weeks); (iii) STING agonist 8803 (5 μg/mouse administered i.e. once per week for 2 weeks); (iv) 8803+IgG control; (v) 8803+anti-PD-1; and (vi) untreated. The QPP8v mice received (i) i.e. vehicle control; (ii) 8803 (5 μg/mouse i.e. on days 7 and 17 after implantation); (iii) 8803+anti-PD-1 (25 μg/mouse i.e. on days 7 and 17); or (iv) 8803+ vehicle control or anti-PD-1 (250 μg i.p. on days 7, 10, and 13) (FIG. 18C). Long-term durable survival was observed with both monotherapeutic STING agonist and in combination with anti-PD-1 (FIG. 18D). More specifically, in the CT-2A glioma model, the control IgG mice had a median survival time of 30 days, 8803-treated mice had a median survival time of 49 days with 50% long-term survivors when the experiment was terminated at 80 days, anti-PD-1-treated mice had a median survival time of 35 days, and the combination had an undefined median survival in which 90% of mice treated with the combination therapy were long-term survivors, which was statistically significant relative to controls and to anti-PD-1 and STING agonist monotherapies (P<0.05). In the QPP8v model, there was no significant treatment benefit of the combination therapy of anti-PD-1 with 8803, including with anti-PD-1 being delivered directly into the glioma (FIG. 18D), suggesting that this combination would likely only be of benefit in scenarios predisposed to respond to immune checkpoint inhibitors. To visualize TME features that would be reflective of response, the CT-2A model was treated on day 7 and during the therapeutic window, the activation of the STING pathway (i.e., p-IRF3) and the immune infiltration were analyzed with multiplexed SeqIF imaging. In the CT-2A model, STING expression was present along the tumor vasculature, but also within the tumor cells, albeit at much less intensity. Forty-eight hours after the first dose of 8803 therapy, there was shrinkage of the tumor size in comparison with control (FIG. 18E). Notably, the p-IRF3 expression was diffusely present. On day 16, 48 hours after the second dose of STING, both CD4+ and CD8+ T cells could be found in the region of the prior tumor (FIG. 18E).

### Example 9

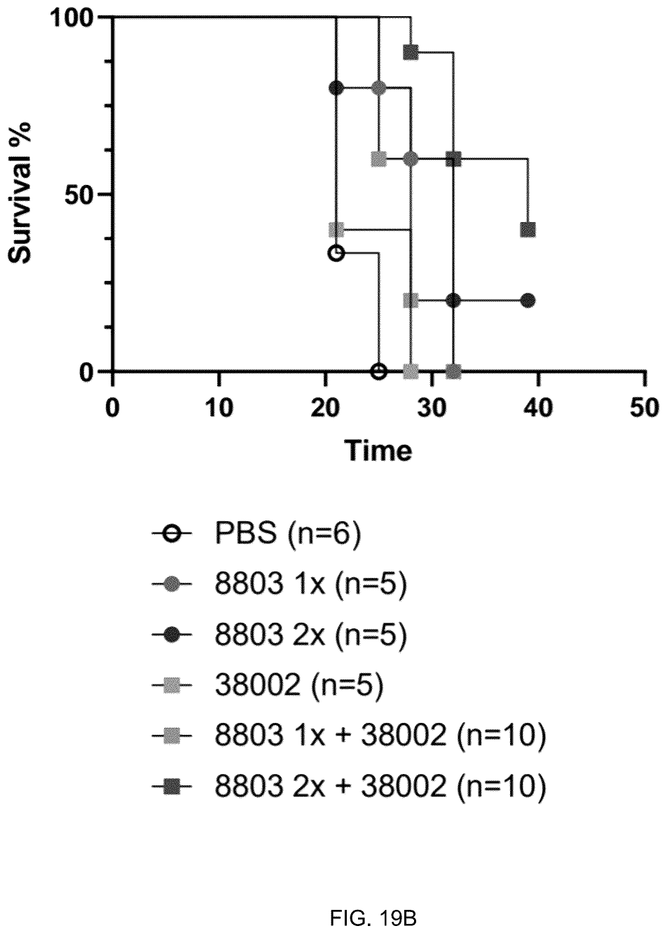

STING agonist 8803 (i.e., Compound A) anti-tumor activity is enhanced by IMGS-001. Animals bearing bilateral flank B16-F10 tumors were treated with the STING agonist 8803 and/or anti-PD-1 (Ab-38002). The mice were randomized to receive (i) anti-PD-1 (10 mg/kg/mouse are administered i.p. 2 times per week for 3 weeks); (ii) STING agonist 8803 (5 μg/mouse are administered i.p. once per week for 2 weeks; (iii) Ab-38002-10 mg/kg, i.p, on day 13, 2 times/week for 3 weeks+STING agonist 8803, 10 mg, i.t., day 13 (3 times per day for 3-4 days) (FIGS. 19A and 19B). Treatment with STING agonist 8803 and Ab-38002 resulted in 40% survival.

It will be readily understood that the components of various embodiments of the present invention, as generally described and illustrated in the figures herein, may be arranged and designed in a wide variety of different configurations. Thus, the detailed description of the embodiments of the present invention, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

What is claimed:

1. A method of treating glioma comprising administering to a subject in need thereof a composition comprising an effective amount of a STING agonist of Formula Ia: 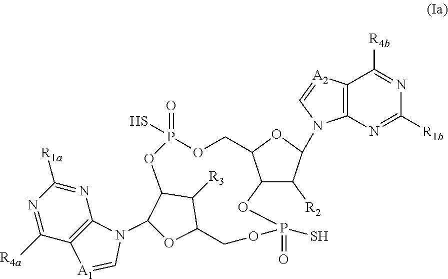
or a salt, ester, tautomer, or prodrug thereof, wherein:
A₁ and A₂ are independently selected from CH and N;
R₁ₐ and R_{1b} are independently selected from H and NH₂;
R₂ is selected from OH, F, CI, N₃, and NH₂;
R₃ is selected from OH, F, Cl, N₃, and NH₂;
R₄ₐ and R_{4b} are independently selected from NH₂, OH, NHR₅, and OR₅; and
R₅ is independently selected from methyl, ethyl, and propyl.

2. The method of claim 1, wherein A₁ is CH and A₂ is N.

3. The method of claim 1, wherein A₁ is N and A₂ is CH.

4. The method of claim 1, wherein A₁ and A₂ are both CH.

5. The method of claim 1, wherein A₁ and A₂ are both N.

6. The method of claim 1, wherein A₁ is N and A₂ is CH, and R₂ is selected from F and Cl, and R₃ is-OH.

7. The method of claim 1, wherein A₁ and A₂ are both N, and R₂ is selected from F and Cl, and R₃ is OH.

8. The method of claim 1, wherein R₁ₐ and R_{1b} are both H.

9. The method of claim 1, wherein R₄ₐ and R_{4b} are independently selected from NH₂ and NHR₅.

10. The method of claim 1, wherein R₄ₐ and R_{4b} are independently selected from NH₂ and NHR₅, R₁ₐ and R_{1b} are both H, A₁ and A₂ are both N, R₂ is selected from F and Cl, and R₃ is OH.

11. The method of claim 1, wherein R₄ₐ and R_{4b} are independently selected from NH₂ and NHR₅, R₁ₐ and R_{1b} are both H, A₁ is N and A₂ is CH, R₂ is selected from F and Cl, and R₃ is OH.

12. The method of claim 1, wherein R₄ₐ and R_{4b} are both NH₂.

13. The method of claim 1, wherein R₄ₐ and R_{4b} are both NH₂, and R₁ₐ and R_{1b} are both H.

14. The method of claim 1, wherein the STING agonist is: 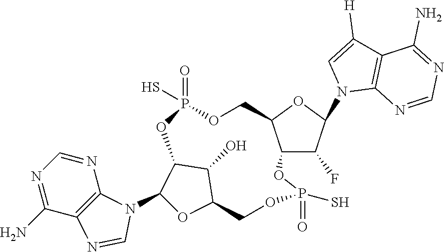 [SMILES: Nc1ncnc2c1ccn2[C@@H]1O[C@@H]2CO[P@@](=O)(S)O[C@@H]3[C@H](O)[C@@H](CO[P@@](=O)(S)O[C@H]2[C@H]1F)O[C@H]3n1cnc2c(N)ncnc21]
or a pharmaceutically acceptable salt thereof.

15. The method of claim 1, wherein the STING agonist is: 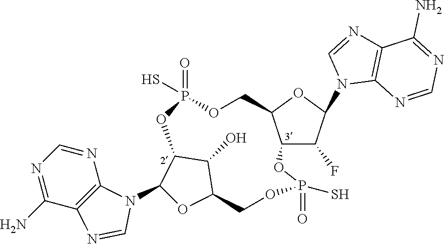 [SMILES: Nc1ncnc2c1ncn2[C@@H]1O[C@@H]2CO[P@@](=O)(S)O[C@@H]3[C@H](O)[C@@H](CO[P@@](=O)(S)O[C@H]2[C@H]1F)O[C@H]3n1cnc2c(N)ncnc21]
or a pharmaceutically acceptable salt thereof.

16. The method of claim 1, wherein the STING agonist is: 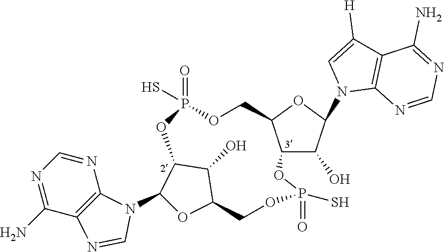 [SMILES: Nc1ncnc2c1ccn2[C@@H]1O[C@@H]2CO[P@@](=O)(S)O[C@@H]3[C@H](O)[C@@H](CO[P@@](=O)(S)O[C@H]2[C@H]1O)O[C@H]3n1cnc2c(N)ncnc21]
or a pharmaceutically acceptable salt thereof.

17. The method of claim 1, wherein the glioma is astrocytomas, ependymomas, or oligodendrogliomas.

18. The method of claim 1, wherein the effective amount is from about 0.005 to about 50 mg/kg body weight.

19. The method of claim 1 wherein the composition is administered with a second therapeutic agent.

20. The method of claim 1 wherein the composition is administered in combination with a chemotherapeutic agent.
